(19) United States
(12) Patent Application Publication
(10) Pub. No.: US 2007/0154533 A1
(43) Pub. Date: Jul. 5, 2007

(54) Method of increasing testosterone and related steriod concentrations in women

(76) Inventor: Robert E. Dudley, Rosemary Beach, FL (US)

Correspondence Address: MAYER, BROWN, ROWE & MAW LLP, 71 S. WACKER, CHICAGO, IL, 60606, US

(21) Appl. No.: 11/402,986

(22) Filed: Apr. 11, 2006

Related U.S. Application Data
(60) Provisional application No. 60/670,753, filed on Apr. 13, 2005.

(51) Int. Cl. A61K 31/57; A61F 13/02

(52) U.S. Cl. 424/448; 514/177

(57) ABSTRACT

The present invention relates to methods, kits, combinations, and compositions for treating, preventing or reducing the risk of developing a testosterone-deficient disorder, or the symptoms associated with, or related to a testosterone-deficient disorder in a subject in need thereof. The present invention also relates to a method of administering a steroid in the testosterone synthetic pathway to a subject in need thereof.

This application claims priority to U.S. provisional Application Ser. No. 60/670,753 filed Apr. 13, 2005, the entire contents of which is hereby incorporated by reference herein.

### FIELD OF THE INVENTION

The present invention is related to methods, kits, combinations, and compositions for transdermally delivering an effective amount of testosterone to a subject in need thereof.

### BACKGROUND OF THE INVENTION

Transdermal preparations of testosterone have provided a useful delivery system for normalizing serum testosterone levels in hypogonadal men and preventing the clinical symptoms and long term effects of androgen deficient men. Available transdermal preparations of testosterone include, for example, TESTODERM®, TESTODERM® TTS, and ANDRODERM®. Testosterone is also available in other formulations including those available as an injectable, for example, DEPO-TESTOSTERONE® (testosterone cypionate), and DELATESTRYL BTG® (testosterone enanthate), or as a gel, for example, ANDROGEL® marketed by Unimed Pharmaceuticals, Inc., Deerfield, Ill., the assignee of this application.

In men, transdermal patches are applied to the scrotal skin or other parts of the body. Recently, a one-percent testosterone gel has been approved for use in men, and provides dosing flexibility with minimal skin irritation. This gel is marketed under the name ANDROGEL®. However, all currently available testosterone transdermal products are specifically contraindicated for use in women in the United States. Furthermore, none of the currently available androgen treatment modalities for women, for example, oral methyltestosterone, intramuscular testosterone ester injections or subcutaneous testosterone implants can achieve reproducible testosterone serum levels on a consistent daily basis.

A. Testosterone Physiology in Women

The excretion of androgenic steroids in the urine of adult women was demonstrated more than 50 years ago. Since that time, physiologists and clinicians have explored the sources and biological functions of testosterone and other endogenous androgenic hormones in the human female, see, for example, Geist S. H., Androgen therapy in the human female, J. Clin. Endocrinol. 1941; 1:154-161. It is now known that androgens are secreted by both the ovaries and adrenal glands in women. Each source contributes about 50% (directly and through precursors) (see, for example, Abraham G. E., Ovarian and adrenal contribution to peripheral androgens during the menstrual cycle, J. Clin. Endocrinol. Metab. 1974; 39:340-346) to the approximately 300 μg of testosterone produced daily in healthy “cycling” women (see, for example, Southren A. L., et al., Further study of factors-affecting the metabolic clearance rate of testosterone in man, J. Clin. Endocrinol. Metab. 1968; 28:1105-1112). While the adverse effects of excess androgen production, as occurs in the polycystic ovary syndrome and certain androgen producing tumors, have been well described (see, for example, Lobo R. A., Chapter 20: Androgen excess in Infertility, Contraception and Reproductive Endocrinology, Third Edition. D R Mishell, V. Davajan and R. Lobo, Editors. Blackwell Scientific Publications, Boston. pp 422-446, 1991), the normal physiological effects of androgens in women have been much less appreciated. As inferred from animal studies, male physiology, and the symptoms of women with deficient androgen production, the major physiological effects of androgens in normal women include, but are not limited to anabolic effects on muscle, skin, hair and bone; stimulatory effects on erythropoiesis; modulatory effects on immune function; and psychological effects on mood, well-being and sexual function.

In addition, endogenous androgens are important for the development of pubic hair and are thought to modulate the action of estrogens and progestins on a variety of reproductive target tissues. It is also believed that androgens play an important role in modulating the secretory function of the lacrimal gland.

Fifty percent of circulating testosterone is derived from direct ovarian secretion in the thecal cells under the control of luteinizing hormone. The other half is derived from peripheral conversion of adrenal androgen precursors dehydroepiandrosterone, androstenedione, and dehydroepiandrosterone sulfate. Testosterone can also be converted to dihydrotestosterone or estradiol. Thus, testosterone serves as both a hormone and as a pro-hormone.

Testosterone circulates in the blood 98% bound to protein. In women, approximately 65% of the binding is to the high-affinity sex hormone binding globulin. The remaining 33% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-sex hormone binding globulin bound testosterone and includes that weakly bound to albumin, as well as that defined as “free.” The order of affinity for the steroids most strongly bound by sex hormone binding globulin is dihydrotestosterone>testosterone>androstenedione>estrogen. Sex hormone binding globulin weakly binds dihydrotestosterone, but not dihydrotestosterone sulfate. Table 1 shows the approximate hormonal levels in normal premenopausal women.

TABLE 1
Hormone Levels in Normal Premenopausal Women

| Hormone | Mean ± sd | Median | Range |
|---|---|---|---|
| Testosterone (nmol/L) | 1.20 ± 0.69 | 0.98 | 0.4-2.7 |
| Free testosterone (pmol/L) | 12.80 ± 5.59 | 12.53 | 4.1-24.2 |
| % Free testosterone of total | 1.4 ± 1.1 | 1.1 | 0.4-6.3 |
| testosterone | | | |
| Luteinizing hormone | 7.2 ± 3.3 | 6.7 | 3.0-18.7 |
| (IU/L) | | | |
| Follicle stimulating | 4.7 ± 3.6 | 4.2 | 1.5-21.4 |
| hormone (IU/L) | | | |
| Sex hormone binding | 66.1 ± 22.7 | 71.0 | 17.8-114.0 |
| globulin (nmol/L) | | | |

However, there is no general consensus on what constitutes “testosterone deficiency” in women because historically it has been impossible to develop assays capable of measuring such small hormonal levels. This is especially true when measuring free or bioavailable testosterone levels. Consequently, currently available laboratory evaluations, including measuring total, free, and bioavailable serum testosterone levels, have not been used extensively to identify hypoandrogenic women.

B. Androgen Administration in Women

In comparison to other hormone deficiency states, testosterone deficiency in women has been largely ignored as a clinical entity, nor has it been defined. Nevertheless, there exist well-defined patient populations where androgen production is clearly deficient and where associated symptomatology has been described, including, for example, young oophorectomized/hysterectomized women, postmenopausal women on estrogen replacement therapy, women on oral contraceptives, women with adrenal dysfunction, women with corticosteroid-induced adrenal suppression, and human immunodeficiency virus-positive women.

Despite the clear benefits of administering testosterone to both normal and testosterone deficient women, almost all of the testosterone delivery preparations for human use are designed for hypogonadal men who require significantly greater amounts of testosterone than a testosterone deficient women. As a result, these formulations and devices are generally unsuitable for women requiring low doses of testosterone. Intramuscular injunction of testosterone esters, for example, is the popular form of androgen replacement for men but is unsatisfactory for women because of the very high levels of testosterone in the first 2-3 days after injection. Moreover, many women report increased acne and occasional cliteromegaly with this type of testosterone administration. Patients receiving injection therapy often complain that the delivery mechanism is painful and causes local skin reactions.

Because increasing testosterone concentrations has been shown to alter sexual performance and libido, researchers have investigated methods of delivering testosterone to men. These methods include intramuscular injections (43%), oral replacement (24%), pellet implants (23%), and transdermal patches (10%). A summary of these methods is shown in Table 2.

TABLE 2
Mode of Application and Dosage of Various Testosterone Preparations

| | Route Of | | |
|---|---|---|---|
| Preparation | Application | Full Substitution Dose | |
| In Clinical Use | | | |
| Testosterone | Intramuscular | 200 | mg every 2-3 weeks |
| enanthate | injection | | |
| Testosterone | Intramuscular | 200 | mg every 2 weeks |
| cypionate | injection | | |
| Testosterone | Oral | 2-4 | capsules at 40 mg per day |
| undecanoate | | | |
| Transdermal | Scrotal skin | 1 | membrane per day |
| testosterone | | | |
| patch | | | |
| Transdermal | Non-scrotal | 1 or 2 | systems per day |
| testosterone | skin | | |
| patch | | | |
| Testosterone | Implantation | 3-6 | implants of 200 mg every |
| implants | under the | | 6 months |
| | abdominal skin | | |
| Under | | | |
| Development | | | |
| Testosterone | Sublingual | 2.5-5 | mg twice daily |
| cyclodextrin | | | |
| Testosterone | Intramuscular | 1000 | mg every 8-10 weeks |
| undecanoate | injection | | |
| Testosterone | Intramuscular | 1000 | mg every 12-16 weeks |
| buciclate | injection | | |
| Testosterone | Intramuscular | 315 | mg for 11 weeks |
| microspheres | injection | | |
| Obsolete | | | |
| 17∝-Methyl- | Oral | 25-5 | g per day |
| testosterone | | | |
| Fluoxymesterone | Sublingual | 10-25 | mg per day |
| | Oral | 10-20 | mg per day |

However, none of the current testosterone replacement products available for use in women are approved in the United States for chronic treatment of the female testosterone deficiency states described herein. Also, currently available methyltestosterone products, which can be administered orally, are no longer recommended as a testosterone replacement method for hypogonadal men, see, for example, Gooren L J. G. and Polderman K. H., Safety aspects of androgens. In Testosterone: Action, Deficiency, Substitution. E. Nieschlag and H M. Behre, editors, Springer-Verlag, Heidelberg, p. 136 (1990). The long acting injectable testosterone-esters, such as enanthate or cypionate are formulated for high dose administration to men (for example 200-300 mg) and produce supra-physiological hormone levels, even when given at lower doses to women (for example 50-100 mg) (see, for example, Sherwin B. B. and Gelfand M. M., Differential symptom response to parenteral estrogen and/or androgen administration in the surgical menopause, Am. J. Obstet. Gynecol. 1985; 151:153-160). Testosterone implants, which have been used experimentally in the past, can likewise produce supra-physiological hormone levels in women, see, for example, Burger H. G. et al., The management of persistent menopausal symptoms with oestradiol-testosterone implants: clinical, lipid and hormonal results, Maturitas 1984; 6:351-358. The supra-physiological androgen levels associated with these products have produced virilizing side effects in some patients, see for example, Burger H. G. et al., (1984). Also see, for example, Sherwin B. B, and Gelfand M. M., (1985). Also see, for example, Urman B., et al., Elevated serum testosterone, hirsutism and virilism associated with combined androgen-estrogen hormone replacement therapy, Obstet. Gynecol., 1991; 7:595-598.

Given the above, however, ESTRATEST®, which is a combination of methyltestosterone and esterified estrogens in oral tablet formulations, is the most commonly used androgen product used to treat women in the United States. At present, however, its only approved indication is for the treatment of moderate to severe vasomotor symptoms associated with menopause in those patients not improved by estrogens alone. Pharmacological doses of methyltestosterone higher than those suggested for hypogonadal men have also been used to treat breast cancer in women. However, oral administration produces inappropriate testosterone levels and unpredictable absorption patterns between patients (Buckler 1998). Moreover, because the liver metabolizes the preparation, there is a risk of hepatoxicity not to mention first pass metabolism.

Testosterone pellet implants (50 mg or 100 mg of testosterone) inserted under local anesthesia in the abdominal wall have been used in conjunction with estrogen pellet implants for many years. Testosterone levels peak about one month after implantation and then return to baseline by month five or six. The testosterone levels are high and characterized by substantial rises and falls over several months and marked individual variation in this period. In addition, implants require a surgical procedure that many men and women simply do not wish to endure. In hypogonadal men, for example, implant therapy includes a risk of extrusion (8.5%), bleeding (2.3%), or infection (0.6%).

Given the problems associated with injected, orally administered and implant-based testosterone delivery methods, researchers have recently begun experimenting with more controlled release preparations that can deliver stable and physiological testosterone levels to women. In the past decade, the transdermal delivery of estradiol has become recognized as a safe, physiological and patient-friendly method for estrogen replacement therapy in women. Second generation estradiol patches that use adhesive matrix technology have recently become available in the United States and Europe. Matrix technology now exists to transdermally administer physiological amounts of testosterone alone for the treatment of androgen deficiency states in women. As the patient populations defined above are approximately 50% deficient in their testosterone production, the transdermal systems have been designed to deliver approximately half of the normal daily testosterone production rate or about 150 μg per day. Matrix technology-based transdermal testosterone administration has been used successfully in women to treat acquired immunodeficiency syndrome wasting and female sexual dysfunction after oophorectomy.

Two testosterone patches for women have been tested in clinical studies. Buckler and his associates have investigated a testosterone patch (Ethical Pharmaceuticals, UK) delivering either 840, 1100, 3000 μg testosterone per day applied twice weekly to the anterior abdominal wall, but did not disclose the composition of the patch (Buckler 1998). Another patch, the TMTDS patch (Watson Laboratories, Salt Lake City, Utah), is a translucent patch having a surface area of 18 cm² which uses sorbitan monooleate as a permeation enhancer and a hypoallergenic acrylic adhesive in an alcohol-free matrix. The average testosterone content of each patch is 4.1 mg. Each patch is designed to deliver testosterone at a nominal rate of 150 g of testosterone per day over an application period of three to four days. Thus, the TMTDS patch is applied twice per week (Javanbakht et al. 2000).

While clinical studies have reported that the testosterone-containing patch is capable of increasing testosterone concentrations in women via a controlled release mechanism, the patches do not provide dosing flexibility. Moreover, their visibility may be esthetically unappealing to some women and may have a tendency to fall off, especially during rigorous physical exercise.

For these and other reasons, therefore, it would be a difficult but much desired advance in the art to provide an effective percutaneously administered testosterone formulation to be applied directly to the skin of a subject, particularly a women, for example, in the form of a gel, ointment, or cream, for example, to treat testosterone-deficient disorders.

### SUMMARY OF THE INVENTION

Pharmaceutical compositions comprising a steroid in the testosterone synthetic pathway have been discovered that can effectively percutaneously deliver a therapeutically-effective amount of the pharmaceutical agent to a female subject. In one embodiment of the present invention, a composition comprising a testosterone dosage amount of about 0.4 mg to about 0.9 mg has been found to possess improved bioavailability, chemical stability, physical stability, safety, as well as other improved pharmacokinetic, pharmacodynamic, chemical and/or physical properties. The present invention comprises these pharmaceutical compositions, dosage forms and kits based thereon, and methods for the preparation and use thereof.

### BRIEF DESCRIPTION OF THE DRAWINGS

FIG. 1 is a graph showing the total testosterone (ng/dL) at baseline prior to administration of either 4.4 mg or 8.8 mg of testosterone in a 1% hydroalcoholic testosterone gel to premenopausal and postmenopausal women.

FIG. 2 is a graph showing the total testosterone (ng/dL) at Day 7 of treatment with administration of either 4.4 mg or 8.8 mg of testosterone in a 1% hydroalcoholic testosterone gel to premenopausal and postmenopausal women.

FIG. 3 is a graph showing the total testosterone (ng/dL) in premenopausal women with administration of 4.4 or 8.8 mg of 1% hydroalcoholic testosterone gel.

FIG. 4 is a graph showing the free testosterone (ng/dL) in premenopausal women with administration of 4.4 or 8.8 mg of 1% hydroalcoholic testosterone gel.

FIG. 5 is a graph showing the total testosterone (ng/dL) in postmenopausal women with administration of 4.4 or 8.8 mg of 1% hydroalcoholic testosterone gel.

FIG. 6 is a graph showing the free testosterone (ng/dL) in postmenopausal women with administration of 4.4 or 8.8 mg of 1% hydroalcoholic testosterone gel.

FIG. 7 is a graph showing the increase in serum testosterone (ng/dL) with administration of either 4.4 mg or 8.8 mg of testosterone in a 1% hydroalcoholic testosterone gel to premenopausal and postmenopausal women.

FIG. 8 is a graph showing total testosterone concentrations during baseline sampling or during administration of different doses of testosterone gel or placebo in postmenopausal women who were not receiving replacement estrogen therapy.

FIG. 9 is a graph showing total testosterone concentrations during baseline sampling or during administration of different doses of testosterone gel or placebo in postmenopausal women who were receiving replacement estrogen therapy.

FIG. 10 is a is a graph showing average testosterone concentrations during baseline sampling or during administration of different doses of testosterone gel or placebo in postmenopausal women who were not receiving replacement estrogen therapy.

FIG. 11 is a graph showing average testosterone concentrations during baseline sampling or during administration of different doses of testosterone gel or placebo in postmenopausal women who were receiving replacement estrogen therapy.

FIG. 12 is a graph showing serum free testosterone concentrations during baseline sampling or during administration of different doses of testosterone gel or placebo in postmenopausal women who were not receiving replacement estrogen therapy.

FIG. 13 is a graph showing serum free testosterone concentrations during baseline sampling or during administration of different doses of testosterone gel or placebo in postmenopausal women who were receiving replacement estrogen therapy.

FIG. 14 is a graph showing estradiol concentrations during baseline sampling or during administration of different doses of testosterone gel in postmenopausal women who were not receiving replacement estrogen therapy.

FIG. 15 is a graph showing estradiol concentrations during baseline sampling or during administration of different doses of testosterone gel in postmenopausal women who were receiving replacement estrogen therapy.

FIG. 16 is a graph showing FSH concentrations during baseline sampling or during administration of different doses of testosterone gel in postmenopausal women who were not receiving replacement estrogen therapy.

FIG. 17 is a graph showing FSH concentrations during baseline sampling or during administration of different doses of testosterone gel in postmenopausal women who were receiving replacement estrogen therapy.

FIG. 18 is a graph showing LH concentrations during baseline sampling or during administration of different doses of testosterone gel in postmenopausal women who were not receiving replacement estrogen therapy.

FIG. 19 is a graph showing LH concentrations during baseline sampling or during administration of different doses of testosterone gel in postmenopausal women who were receiving replacement estrogen therapy.

FIG. 20 is a graph showing SHBG concentrations during baseline sampling or during administration of different doses of testosterone gel in postmenopausal women who were not receiving replacement estrogen therapy.

FIG. 21 is a graph showing SHBG concentrations during baseline sampling or during administration of different doses of testosterone gel in postmenopausal women who were receiving replacement estrogen therapy.

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated. The methods, kits, combinations, and compositions of the present invention provide enhanced treatment options for treating a testosterone-deficient disorder in a subject, for example, a women, as compared to those currently available.

Where the invention is illustrated herein with particular reference to testosterone, it will be understood that any other steroid in the testosterone synthetic pathway can, if desired, be substituted in whole or in part for testosterone in the methods, kits, combinations, and compositions herein described. Where the invention is illustrated herein with particular reference to methyltestosterone, it will be understood that any other inhibitor of the synthesis of sex hormone binding globulin can, if desired, be substituted in whole or in part for methyltestosterone in the methods, kits, combinations, and compositions herein described. Where the invention is illustrated herein with particular reference, to estradiol, it will be understood that any other estrogenic hormone can, if desired, be substituted in whole or in part for estradiol in the methods, kits, combinations, and compositions herein described.

The present invention is directed to methods, kits, combinations, and compositions for treating, preventing or reducing the risk of developing a testosterone-deficient disorder, or the symptoms associated with, or related to a testosterone-deficient disorder in a subject in need thereof. In one embodiment of the present invention the subject is female. The method comprises percutaneously administering a testosterone-deficient disorder-effective amount of a steroid in the testosterone synthetic pathway, for example, testosterone, to a subject. The present invention includes methods of reversing, halting or slowing the progression of a testosterone-deficient disorder once it becomes clinically evident, or treating the symptoms associated with, or related to the testosterone-deficient disorder. The subject may already have a testosterone-deficient disorder at the time of administration, or be at risk of developing a testosterone-deficient disorder. Also included in the present invention is a method of administering a steroid in the testosterone synthetic pathway, for example testosterone, to a mammal in need thereof. The method comprises administering to the subject a testosterone-deficient disorder-effective amount of a percutaneously deliverable composition comprised of a pharmaceutically-acceptable steroid in the testosterone synthetic pathway, for example testosterone, one or more lower alcohols, such as ethanol or isopropanol, a penetration enhancing agent, a thickener, and water. Also included in the methods, kits, combinations, and compositions of the present invention are pharmaceutical compositions comprising a testosterone-deficient disorder-effective amount of testosterone. In one embodiment the testosterone composition is formulated as a hydroalcoholic gel. In another embodiment, the gel comprises testosterone, one or more lower alcohols, such as ethanol or isopropanol, a penetration enhancing agent, a thickener, and water. The present invention also includes kits comprising percutaneously deliverable testosterone. The kits may also contain a set of instructions for the patient.

In another embodiment, the methods, kits, combinations, and compositions are used in conjunction with other steroids or pharmaceutical agents effective at treating, preventing, or reducing the risk of developing a testosterone deficient disorder in a subject. In one embodiment, the present invention employing testosterone is used in conjunction with a pharmacologically effective amount of an estrogenic hormone, for example, estradiol either in the same dosage form or as separate dosage forms. In another embodiment, the methods, kits, combinations, and compositions are used with another steroid or pharmaceutical agent that increases testosterone levels in a mammal, for example, testosterone. Additionally, the present invention optionally includes salts, esters, amides, enantiomers, isomers, tautomers, prodrugs, or derivatives of the compounds of the present inventions, as well as emollients, stabilizers, antimicrobials, fragrances, and propellants. The methods, kits, combinations, and compositions of the present invention provide enhanced treatment options for treating a testosterone deficient disorder in a subject, for example, a woman, as compared to those currently available.

Besides being useful for human treatment, the present invention is also useful for other subjects including veterinary animals, reptiles, birds, exotic animals and farm animals, including mammals, rodents, and the like. Mammals include primates, for example, a monkey, or a lemur, horses, dogs, pigs, or cats. Rodents includes rats, mice, squirrels, or guinea pigs.

The use of the phrase “adult female premenopausal subject” refers to a fully grown and mature female mammal, for example, a human, capable of menstruation including undergoing cyclic endometrial shedding and discharge of bloody fluid from the uterus during the menstrual cycle. In a healthy subject, this cyclic shedding and discharge occurs at regular intervals, for example, approximately every four weeks in a human, and under normal circumstances is preceded by ovulation and predecidual changes in the endometrium. (See, for example, Stedman's Medial Dictionary, 25^{th} Edition).

The use of the phrase “adult female postmenopausal subject” refers to a fully grown mature female mammal, for example, a human, that has undergone menopause, or in other word, is no longer capable of menses due to permanent cessation of menstrual cycling. (See, for example, Stedman's Medial Dictionary, 25^{th} Edition).

The use of the phrase “below-normal free testosterone plasma concentration” refers to an adult female subject, being either premenopausal or postmenopausal, having a free testosterone plasma concentration of less than about 0.2 ng per dL serum to about 0.5 ng per dL serum.

The term “treat” or “treatment” as used herein refers to any treatment of a mammalian condition, disorder, or disease associated with an androgen deficiency or a testosterone deficiency, and includes, but is not limited to, preventing the condition, disorder, or disease from occurring in a mammal which may be predisposed to the condition, disorder, or disease, but has not yet been diagnosed as having the condition, disorder, or disease; inhibiting the condition, disorder, or disease, for example, arresting the development of the condition, disorder, or disease; relieving the condition, disorder, or disease, for example, causing regression of the condition, disorder, or disease; or relieving the condition caused by the disease or disorder, for example, stopping the symptoms of the disease or disorder.

The term “prevent” or “prevention,” in relation to a testosterone deficient condition, disorder, or disease, means no testosterone deficient condition, disorder, or disease development if none had occurred, or no further testosterone deficient condition, disorder, or disease development if there had already been development of the testosterone deficient condition, disorder, or disease.

The phrase “testosterone-deficient disorder” refers to a to a condition, disorder, or disease that occurs in a mammal due to lack of endogenous testosterone production or utilization thereof. In women, such conditions, disorders, or diseases include, but are not limited to, hypogonadism, sexual dysfunction (including, for example, sexual desire disorders, sexual arousal disorders, orgasmic disorders (including, for example, persistent or routine inability to attain or maintain genital lubrication or engorgement in response to sexual stimulation), and sexual pain disorders), decreased libido, hypercholesterolemia, abnormal electrocardiograms, vasomotor symptoms, diabetic retinopathy, hyperglycemia, hyperinsulinemia, hypoinsulinemia, increased percentage of body fat, hypertension, obesity, osteoporosis, osteopenia, vaginal dryness, thinning of the vaginal wall, menopausal symptoms and hot flashes, cognitive dysfunction, cardiovascular disease, central nervous system disorders, Alzheimer's disease, dementia, cataracts, cervical cancer, uterine cancer, breast cancer, depression (including, for example, postpartum depression), in vitro fertilization (including, for example, improper embryo implantation in the uterus), premature ovarian failure, and premature menopause.

As used herein, the phrase “therapeutic-effective amount,” means an amount effective to deliver sufficient a therapeutic agent on the present invention to achieve a desired therapeutic result in the treatment of a condition. The amount that constitutes a therapeutically effective amount varies according to the condition being treated (for example, hypogonadism, sexual dysfunction, decreased libido, hypercholesterolemia, abnormal electrocardiograms, vasomotor symptoms, diabetic retinopathy, hyperglycemia, hyperinsulinemia, hypoinsulinemia, increased percentage of body fat, hypertension, obesity, osteoporosis, osteopenia, vaginal dryness, thinning of the vaginal wall, menopausal symptoms and hot flashes, cognitive dysfunction, cardiovascular disease, central nervous system disorders, Alzheimer's disease, dementia, dementia, cataracts, cervical cancer, uterine cancer, breast cancer, depression (including, for example, postpartum depression), in vitro fertilization (including, for example, improper embryo implantation in the uterus), premature ovarian failure, and premature menopause, any drugs being coadministered with therapeutic agent, desired duration of treatment, the surface area and location of the skin over which the composition is administered, and the selection of adjuvant and other components of the composition. Accordingly, it is not practical to enumerate particular illustrative amounts but such can be readily determined by those skilled in the art with due consideration of these and other appropriate factors, however, several non-limiting examples are provided herein for illustrative purposes.

As used herein, the phrases “androgen deficiency” or “testosterone deficiency” are used interchangeably, and refer to lower serum levels of free testosterone in a subject as compared to the median serum levels for healthy women of the same age. Normal cycling women produce approximately 300 μg of testosterone per day. Their total serum testosterone levels generally range from about 20 ng/dL to about 80 ng/dL averaging about 40 ng/dL. In healthy young women, for example, mean free testosterone levels are generally about 3.6 pg/mL. However, several factors may influence both total and free testosterone serum levels. For example, in regularly ovulating women, there is a small but significant increase in plasma testosterone levels during the middle third of the menstrual cycle. However, mean testosterone levels (1.2 nmol/L or 33 ng/dL) and mean free testosterone levels (12.8 pmol/L or 3.6 pg/mL) during the luteal and follicular phases are not significantly different. Additionally, testosterone production declines continuously after age 30 so that serum testosterone levels in a 60-year-old woman are only 50% of the levels in a young 30-year-old woman. Although the percentage of free testosterone generally does not vary with age, an absolute decline in free testosterone has been observed. This decline does not occur abruptly at menopause but instead occurs gradually and continuously as a result of the age-related decrease in both the adrenal and ovarian androgen production. Thus, women begin to experience symptoms associated with menopause in the immediate premenopausal years. The decline in testosterone following menopause results from the combination of ovarian failure, decreasing renal secretion, and peripheral conversion. Also, for example, after ovariectomy, testosterone concentrations decrease by about 50%. Signs or symptoms of testosterone deficiency include, for example, bone loss, dysphoria or diminished sense of well-being, decreased muscle strength, fatigue, decreased libido, decreased sexual receptivity and pleasure, and changes in cognition or memory.

A “testosterone-deficient-disorder effect” or “testosterone-deficient-disorder-effective amount” is intended to qualify the amount of testosterone required to treat or prevent a testosterone-deficient disorder in a mammal, or relieve to some extent one or more of the symptoms associated with, or related to, a testosterone-deficient disorder in a mammal. In a woman, this includes, but is not limited to, normalizing hypogonadism; improving sexual dysfunction; increasing libido; normalizing cholesterol levels; normalizing abnormal electrocardiograms of patients and improving vasomotor symptoms; improving diabetic retinopathy as well as lowering the insulin requirements of diabetic patients; decreasing the percentage of body fat; normalizing glucose levels; decreasing the risk factors for cardiovascular disease, including normalizing hypertension, and treating obesity; preventing osteoporosis, osteopenia, vaginal dryness, and thinning of the vaginal wall; relieving menopausal symptoms and hot flashes; improving cognitive dysfunction; treating, preventing or reducing the onset of cardiovascular disease, Alzheimer's disease, dementia, and cataracts; treating, preventing or reducing the risk of cervical, uterine or breast cancer; treating, preventing or reducing the risk depression, including, for example, postpartum depression; improving the effectiveness or reducing the risk of failure in in vitro fertilization techniques, such as, improper embryo implantation in the uterus; treating, preventing or reducing the risk of developing premature menopause.

The use of the term “about” in the present disclosure means “approximately,” and use of the term “about” indicates that dosages slightly outside the cited ranges may also be effective and safe, and such dosages are also encompassed by the scope of the present claims.

The phrase “pharmaceutically acceptable” is used adjectivally herein to mean that the modified noun is appropriate for use in a pharmaceutical product. Pharmaceutically acceptable cations include metallic ions and organic ions. Illustratively, metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Illustratively, organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The phrase “penetration enhancer” refers to an agent known to accelerate the delivery of the drug through the skin. These agents also have been referred to as accelerants, adjuvants, and absorption promoters, and are collectively referred to herein as “enhancers.” This class of agents includes those with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer. The penetration enhancer of the present invention is a functional derivative of a fatty acid, which includes isosteric modifications of fatty acids or non-acidic derivatives of the carboxylic functional group of a fatty acid or isosteric modifications thereof. In one embodiment, the functional derivative of a fatty acid is an unsaturated alkanoic acid in which the —COOH group is substituted with a functional derivative thereof, such as alcohols, polyols, amides and substituted derivatives thereof. The term “fatty acid” means a fatty acid that has four (4) to twenty-four 24) carbon atoms.

Non-limiting examples of penetration enhancers include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickeners used herein may include anionic polymers such as polyacrylic acid (CARBOPOL® by B.F. Goodrich Specialty Polymers and Chemicals Division of Cleveland, Ohio), carboxymethylcellulose and the like. Additional thickeners, enhancers and adjuvants may generally be found in Remington's The Science and Practice of Pharmacy, Meade Publishing Co., United States Pharmacopeia/National Formulary.

As used herein, the term “lower alcohol,” alone or in combination, means a straight-chain or branched-chain alcohol moiety containing one to about six carbon atoms. In one embodiment, the lower alcohol contains one to about 4 carbon atoms, and in another embodiment the lower alcohol contains two to about 3 carbon atoms. Examples of such alcohol moieties include methanol, ethanol, n-propanol, isopropanol, n-butanol, isobutanol, sec-butanol, and tert-butanol.

As used herein, the term “lower alkyl”, alone or in combination, means a straight-chain or branched-chain alkyl radical containing one to about six carbon atoms. In one embodiment, the lower alkyl contains one to about four carbon atoms. Examples of such radicals include methyl, ethyl, n-propyl, isopropyl, n-butyl, isobutyl, sec-butyl, and tert-butyl.

The compositions of the present invention are used in a “testosterone-deficient-disorder-effective amount.” This means that the concentration of the testosterone is such that a therapeutic level of drug is delivered over the term that the percutaneously delivered formulation is to be used. Such delivery is dependent on a number of variables including the time period for which the individual dosage unit is to be used, the flux rate of the therapeutic agent, for example, testosterone, from the gel, surface area of application site, etc. The amount of therapeutic agent necessary can be experimentally determined based on the flux rate of the drug through the gel, and through the skin when used with and without enhancers. It is understood, however, that specific dose levels of the therapeutic agents of the present invention for any particular patient depends upon a variety of factors including the activity of the specific compound employed, the age, body weight, general health, sex, and diet of the patient, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of menopause in accordance with the present invention. In terms of treatment protocols, it should be appreciated that the dosage to be administered will depend on several factors, including the particular agent that is administered, the route administered the condition of the particular patient, etc. Generally speaking, one will desire to administer an amount of the compound that is effective to achieve a serum level commensurate with the concentrations found to be effective in vitro, assuming that such test have predictive in vivo values. Thus, where an compound is found to demonstrate in vitro activity at, for example, 10 ng/ml, one will desire to administer an amount of the drug that is effective to provide about a 10 ng/ml concentration in vivo. Determination of these parameters is well within the skill of the art. These considerations, as well as effective formulations and administration procedures are well known in the art and are described in standard textbooks.

In order to measure and determine the testosterone deficient-effective amount of testosterone to be delivered to a subject, serum testosterone concentrations can be measured using standard assay techniques. For example, free serum testosterone levels can be measured by the recently validated and highly sensitive equilibrium dialysis method discussed in Sinha-Hikim et al., The Use of a Sensitive Equilibrium Dialysis Method for the Measurement of Free Testosterone Levels in Healthy, Cycling Women and in HIV-Infected Women, 83 J. CLINICAL ENDOCRINOLOGY & METABOLISM 1312-18. (1998), and is herein fully incorporated by reference.

In one embodiment of the present invention, a method of treating, preventing or reducing the risk of developing a testosterone-deficient disorder in a female subject in need thereof is provided. The method comprises administering an amount of a composition to an area (generally greater than about 5 square centimeters) of skin of the subject, which delivers a therapeutically-effective amount of testosterone to the blood serum of the subject. The composition can comprise about 0.1% to about 10% testosterone, or a salt, an ester, a hydrate, an amide, an enantiomer, an isomer, a tautomer, a polymorph, a prodrug, a derivative thereof; about 30% to about 98% alcohol selected from the group consisting of ethanol or isopropanol; about 0.1% to about 5% isopropyl myristate; about 0.1% to about 5% of a gelling agent; and the balance water. The percentages of the composition are on a weight to weight basis of the composition and the sum of components of the composition is about 100 weight percent. The composition is capable of releasing the testosterone to the skin at a rate and duration that raises testosterone blood serum concentration to at least about 3 pg testosterone/ml blood serum within about 24 hours after administration. In one embodiment, the composition comprises about 1% to about 10% 0.1 N sodium hydroxide.

In one embodiment of the present invention, the gelling agent is selected from the group consisting of polyacrylic acid and carboxymethylcellulose. In one embodiment, the gelling agent is polyacrylic acid present in an amount of about 1% weight to weight of the composition.

In another embodiment of the present invention, the composition weighs less than or equal to about 100 grams. In yet another embodiment, the composition weighs about 1 grams to about 10 grams. And in another embodiment, the composition weighs about 4.4 grams to about 8.8 grams. And in yet another embodiment, the composition weighs about 2.5 grams to about 7.5 grams.

In yet another embodiment of the present invention, the composition is the form of a gel.

In still another embodiment of the present invention, for each about 0.1 gram per day application of the composition to the skin, an increase of at least about 5 ng/dL in serum testosterone concentration results in the subject.

In yet another embodiment of the present invention, the composition is provided to the subject for daily administration in about a 0.1 g to about a 10 g dose. In another embodiment, the amount of the composition is a 0.44 g dose delivering about 0.44 mg to about 44 mg of testosterone to the skin. And in yet another the amount of the composition is a 0.44 g dose delivering about 2.2 mg to about 4.4 mg of testosterone to the skin. In another embodiment, the amount of the composition is a 8.8 g dose delivering about 0.88 mg to about 88 mg of testosterone to the skin. And in still another embodiment, the amount of the composition is a 0.88 g dose delivering 4.4 mg to about 8.8 mg of testosterone to the skin. In another embodiment, the amount of the composition is a 1.32 g dose delivering about 1.32 mg to about 132 mg of testosterone to the skin. And in still another embodiment, the amount of the composition is a 1.32 g dose delivering 6.6 mg to about 13.2 mg of testosterone to the skin.

In one embodiment of the present invention, administration of the composition results in a steady-state testosterone level by day 3 of treatment.

In another embodiment of the present invention, the composition is provided to the subject in one or more packets, which can comprises a polyethylene liner between the composition and inner surface of the packet. The packet may hold a unit dose or multiple dose.

In another embodiment, the composition is dispensed from a rigid multi-dose container (for example, with a hand pump) having a larger foil packet, for example, of the composition inside the container. Such larger packets can also comprise a polyethylene liner as above. In one embodiment, the multi-dose container comprises an airless pump that comprises a polyethylene pouch within a canister with a hand pump inserted. In one embodiment, the polyethylene pouch comprises 44 g or 88 g of product. In one embodiment, the pump is primed before use, such as, e.g., by fully depressing the pump three times and discarding the gel. In one embodiment, the pump contains enough product to allow for priming and a set number of precise doses. In one embodiment, each full pump depression delivers 1.25 g of testosterone gel. In this embodiment, a 3.75 g dose of gel would require 3 pump depressions. A 5 g dose of gel would require 4 pump depressions. A 7.5 g dose of gel would require 6 pump depressions. A 10 g dose of gel would require 8 depressions, and so on. Of course, each pump depression can deliver any amount of testosterone gel suitable for delivering the desired dose.

In yet another embodiment of the present invention, the composition is provided as a separate component to a kit.

In still another embodiment of the present invention, the composition is administered once, twice, or three times a day, or as many times necessary to achieve a therapeutic effect.

In another embodiment of the present invention, the composition further comprises about 0.01% to about 69% of a therapeutic agent comprising an agent that inhibits the synthesis of the sex hormone binding globulin, a progesterone, a progestin, or an estrogenic hormone. In yet another embodiment, the therapeutic agent comprises about 1% to about 10% of the composition. In still another embodiment of the present invention, the therapeutic agent is progesterone. And in another embodiment the serum blood level of progesterone is raised to at least about 1 ng progesterone/ml blood serum within about 24 hours after administration of the composition to the subject. In still another embodiment of the present invention, the therapeutic agent is estrogen. And in another embodiment, the serum blood level of estrogen is raised to at least 60 pg estrogen/ml blood serum within about 24 hours after administration.

In one embodiment of the present invention, a method of treating, preventing or reducing the risk of developing a testosterone-deficient disorder in a female subject in need thereof, is provided, that comprises identifying a female subject having, or at risk of developing, a testosterone-deficient disorder; and administering an amount of a composition to an area of skin of the subject, which delivers a therapeutically-effective amount of testosterone to the blood serum of the subject such that the testosterone-deficient disorder or the risk of developing a testosterone-deficient disorder is reduced. In one embodiment, the composition comprises about 0.1% to 10% testosterone, or a salt, ester, amide, enantiomer, isomer, tautomer, prodrug, or derivative thereof, about 30% to about 98% alcohol selected from the group consisting of ethanol or isopropanol; about 0.1% to about 5% of a gelling agent; and the balance water; wherein the percentages are on a weight to weight basis of the composition and the sum of components of the composition is about 100 weight %. In one embodiment, the sum of the components is less than 100% and water is added in a quantity sufficient to make 100%. In one embodiment, the composition is capable of releasing the testosterone to the skin at a rate and duration that raises testosterone blood serum concentration to at least about 3 pg testosterone/ml blood serum within about 24 hours after administration. In one embodiment, the composition comprises about 1% to about 10% 0.1 N sodium hydroxide.

In another embodiment of the present invention, a method of delivering a testosterone-deficient disorder-effective amount of testosterone to blood serum of a female subject in need thereof is provided, which comprises contacting the skin of the subject with a composition comprising about 0.1% to about 10% testosterone, or a salt, an ester, a hydrate, an amide, an enantiomer, an isomer, a tautomer, a polymorph, a prodrug, a derivative thereof; about 30% to about 98% alcohol selected from the group consisting of ethanol or isopropanol; about 0.1% to about 5% isopropyl myristate; about 0.1% to about 10% 0.1 N sodium hydroxide; about 0.1% to about 5% of a gelling agent; and the balance water; wherein the percentages are on a weight to weight basis of the composition and the sum of components of the composition is about 100 weight %. In one embodiment, the sum of the components is less than 100% and water is added in a quantity sufficient to make 100%. In one embodiment, the composition is capable of releasing the testosterone to the skin at a rate and duration that raises testosterone blood serum concentration to at least about 3 pg testosterone/ml blood serum within about 24 hours after administration.

In yet another embodiment of the present invention, a method for administering a testosterone-deficient-disorder-effective amount of testosterone to blood serum of a female subject in need thereof, is provided, the method comprises providing a pharmaceutical composition comprising about 0.1% to about 10% testosterone, or a salt, an ester, a hydrate, an amide, an enantiomer, an isomer, a tautomer, a polymorph, a prodrug, a derivative thereof; about 30% to about 98% alcohol selected from the group consisting of ethanol or isopropanol; about 0.1% to about 5% isopropyl myristate; about 0.1% to about 10% 0.1 N sodium hydroxide; and about 0.1% to about 5% of a gelling agent; and applying the composition to skin of the subject in an amount sufficient for the testosterone to reach the blood serum of the subject so as to achieve a serum concentration of at least 3 pg testosterone/ml blood serum within about 24 hours after administration. The percentages are on a weight to weight basis of the composition and the sum of components of the composition is about 100 weight %. In one embodiment, the sum of the components is less than 100% and water is added in a quantity sufficient to make 100%.

A class of steroids in the testosterone synthetic pathway useful in the methods, kits, combinations, and compositions of the present invention include steroids in the testosterone anabolic or catabolic pathway. In a broad aspect of the invention, the active ingredients employed in the composition may include anabolic steroids such as androisoxazole, bolasterone, clostebol, ethylestrenol, formyldienolone, 4-hydroxy-19-nortestosterone, methenolone, methyltrienolone, nandrolone, oxymesterone, quinbolone, stenbolone, trenbolone; androgenic steroids such as boldenone, fluoxymesterone, mestanolone, mesterolone, methandrostenolone, 17∝methyltestosterone, 17 alpha-methyl-testosterone 3-cyclopentyl enol ether, norethandrolone, normethandrone, oxandrolone, oxymetholone, prasterone, stanlolone, stanozolol, dihydrotestosterone, testosterone; and progestogens such as anagestone, chlormadinone acetate, delmadinone acetate, demegestone, dimethisterone, dihydrogesterone, ethinylestrenol, ethisterone, ethynodiol, ethynodiol diacetate, flurogestone acetate, gestodene, gestonorone caproate, haloprogesterone, 17-hydroxy-16-methyleneprogesterone, 17 alpha-hydroxyprogesterone, 17 alpha-hydroxyprogesterone caproate, medrogestone, medroxyprogesterone, megestrol acetate, melengestrol, norethindrone, norethindrone acetate, norethynodrel, norgesterone, norgestimate, norgestrel, norgestrienone, 19-norprogesterone, norvinisterone, pentagestrone, progesterone, promegestone, quingestrone, and trengestone; and all salts, esters, hydrates, amides, enantiomers, isomers, tautomers, polymorphs, prodrugs, or derivatives of these compounds. (Based upon the list provided in The Merck Index, Merck & Co. Rahway, N.J. (1998)). Combinations of the above mentioned steroids can be used.

In one embodiment, testosterone is formulated as a hydroalcoholic gel. In another embodiment, the gel comprises testosterone, one or more lower alcohols, such as ethanol or isopropanol, a penetration enhancing agent, a thickener (aka a gelling agent), and water. In one embodiment, the gel further comprises a hydroxide releasing agent, such as, e.g, sodium hydroxide. Additionally, the present invention may optionally include salts, emollients, stabilizers, antimicrobials, fragrances, and propellants.

Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol; diethylene-glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickening agents (aka gelling agents) used herein may include anionic polymers such as polyacrylic acid (CARBOPOL® by B.F. Goodrich Specialty Polymers and Chemicals Division of Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, and other polymers such as Pemulen® polymeric emulsifiers, and Noveon® polycarbophils. Additional thickening agents, enhancers and adjuvants may generally be found in Remington's The Science and Practice of Pharmacy, Meade Publishing Co., United States Pharmacopeia/National Formulary.

In one embodiment, the formulation of the present invention delivers about 0.5 mg to about 50 mg testosterone, or the equivalent thereof, to a subject per dosage unit. In another embodiment of the present invention, the formulation delivers from about 0.5 mg to about 25 mg testosterone, or the equivalent thereof, to a subject per dosage unit. In yet another embodiment of the present invention, the formulations of the present invention deliver from about 4 mg to about 15 mg testosterone, or the equivalent thereof, to a subject per dosage unit. In another embodiment of the present invention, the formulations of the present invention deliver about 4.4 mg testosterone, or about 8.8 gm testosterone, or about 13.2 mg testosterone, or the equivalent thereof, to a subject per dosage unit. Thus, for example, a testosterone gel, ointment, cream or patch formulated for once a day administration can contain about 4.4 mg, or about 8.8 mg, or about 13.2 mg testosterone.

In one embodiment, the formulation is a gel, an ointment, a cream or a patch and is comprised of testosterone; a penetration enhancing agent, such as isopropyl myristate; a thickening agent, such as Carbopol; a lower alcohol, such as ethanol or isopropanol; and water. In another embodiment the formulation is a gel, an ointment, a cream or a patch and is comprised of the following substances in approximate percentages:

TABLE 3
Composition of Testosterone Formulation

| SUBSTANCE | AMOUNT (w/w) |
|---|---|
| Testosterone | 0.01-15% |
| Penetration | 0.01-50% |
| enhancing agent | |
| Gelling agent | 0.01-50% |
| Lower alcohol | 30-98% |
| Purified water (qs) | to 100% |

In one embodiment, in a 100 g composition, the gel, ointment, cream, or patch may contain about 0.01 g to about 15 g of testosterone, about 0.01 g to about 50 g penetration enhancing agent, about 0.1 g to about 50 g gelling agent, and about 30 g to about 98 g lower alcohol. In another embodiment, in a 100 g composition, the gel, ointment, cream, or patch may contain about 0.1 g to 10 g of testosterone, about 0.1 g to about 5 g of penetration enhancing agent, about 0.1 g to about 5 g of gelling agent, an about 45 g to about 90 g lower alcohol.

In one embodiment, the composition is a gel, ointment, cream, or patch that further comprises sodium hydroxide or triethanolamine or potassium hydroxide, or a combination thereof, in an amount sufficient, as is known in the art, to assist the gelling agent in forming a gel. In one embodiment, a solution of sodium hydroxide is used, such as, e.g., 0.1 N sodium hydroxide solution, 0.2 N sodium hydroxide solution, 0.5 N sodium hydroxide solution, 1.0 N sodium hydroxide solution, 1.5 N sodium hydroxide solution, 2.0 N sodium hydroxide solution, or any other suitable solution for providing an amount sufficient of the sodium hydroxide to the composition. In one embodiment, the composition comprises about 1% to about 10% 0.1 N sodium hydroxide.

In another embodiment, the pharmaceutical composition includes about 0.5% to about 10% testosterone; about 30% to about 98% alcohol, for example, ethanol or isopropanol; about 0.1% to about 5% isopropyl myristate; about 0.1% to about 5% of a gelling agent; and the balance water. The percentages of components are weight to weight of the composition.

In yet another, embodiment, the pharmaceutical composition includes testosterone in a hydroalcoholic gel. The testosterone may be present in a concentration of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 10.1%, about 10.2%, about 10.3%, about 10.4%, about 10.5%, about 10.6%, about 10.7%, about 10.8%, about 10.9%, about 11%, about 11.1%, about 11.2%, about 11.3%, about 11.4%, about 11.5%, about 11.6%, about 11.7%, about 11.8%, about 11.9%, about 12%, about 12.1%, about 12.2%, about 12.3%, about 12.4%, about 12.5%, about 12.6%, about 12.7%, about 12.8%, about 12.9%, about 13%, about 13.1%, about 13.2%, about 13.3%, about 13.4%, about 13.5%, about 13.6%, about 13.7%, about 13.8%, about 13.9%, about 14%, about 14.1%, about 14.2%, about 14.3%, about 14.4%, about 14.5%, about 14.6%, about 14.7%, about 14.8%, about 14.9%, or about 15% weight to weight of the composition. The enhancer in this embodiment includes isopropyl myristate, which may be present in a concentration of about 0.5%, about 0.65%, about 0.75%, about 0.85%, about 0.95%, about 1%, about 2%, about 3%, about 4%, or about 5% weight to weight of the composition. The pharmaceutical composition also includes a C1-C4 alcohol present in a concentration of about 70%, about 71%, about 71.4%, about 71.8%, about 72%, about 72.3%, about 72.5%, about 72.7%, about 73%, about 73.5%, about 74%, about 74.5%, about 75% or about 75% weight to weight of the composition. Further, the pharmaceutical composition includes polyacrylic acid and/or carboxymethylcellulose as the gelling agent. In one embodiment, the gelling agent is polyacrylic acid present in a concentration of about 1% weight to weight of the composition.

In one embodiment, the gel is comprised of the following substances in approximate amounts:

TABLE 4
Composition of ReLibra ®

| | AMOUNT (w/w) | |
|---|---|---|
| | PER 100 g | |
| SUBSTANCE | OF GEL | |
| Testosterone | 1.0 | g |
| Carbopol 980 | 0.90 | g |
| Isopropyl myristate | 0.50 | g |
| 0.1 N NaOH | 4.72 | g |
| Ethanol (96% v/v) | 71.4 | g* |
| Purified water (qs) | to 100 | g |

*Corresponding to 67 g of ethanol

One skilled in the art will appreciate that the constituents of this formulation may be varied in amounts yet continue to be within the spirit and scope of the present invention. For example, the composition may contain about 0.1 to about 10.0 g of testosterone, about 0.1 to about 5.0 g CARBOPOL, about 0.1 to about 5.0 g isopropyl myristate, about 30.0 to about 98.0 g ethanol, and the balance water.

In still another embodiment, the composition comprises testosterone in an amount greater than 0.01%, a penetration enhancing agent in an amount greater than about 0.1%, a thickening agent in an amount greater than about 0.1%, and a lower alcohol in an amount greater than about 30% w/w of the composition. The gel is rubbed onto the clean dry skin of the upper outer thigh and hip once daily. Following application, the gel is allowed to air dry. The patient washes her hands. Application of the gel results in an increased testosterone level having a desirable pharmacokinetic profile similar to that in normal women. The gel is thus useful for treating a number of conditions or diseases in women.

In one embodiment, about 0.44 g of gel is applied to the skin of the subject, delivering about 4.4 mg of testosterone to the skin. In one embodiment, about 0.88 g of gel is applied to the skin of the subject, delivering about 8.8 mg of testosterone to the skin. In another embodiment, about 1.32 g of gel is applied to the skin of the subject delivering about 13.2 mg of testosterone to the skin.

Achieving target delivery rates demonstrated by testosterone gel can be estimated from the pharmacokinetics in testosterone gel in men. The mean serum concentration (C_{avg}) values in men after applying of varying amounts of gel to the upper body is given below in Table 5.

TABLE 5
Mean Average Serum Testosterone Concentrations and Daily Delivery
Rate after Administration of Testosterone Gel 1% in Men

| Dose (μL) | Mean Cavg | Daily Delivery Rate |
|---|---|---|
| (gram) | (ng/dL) | (μg/day)^{a} |
| 5.0 | 555 (±225) | 3330 |
| 7.5 | 601 (±309) | 3606 |
| 10.0 | 713 (±209) | 4278 |

^{a}Metabolic Clearance Rate of Daily Testosterone = 600 L/day

Based on the results obtained in men, a testosterone gel dose of 0.5 grams delivers approximately 300 μg of testosterone per day.

Illustratively, for an adult woman, a testosterone-deficient disorder-effective amount of testosterone per daily dose delivers to the blood serum typically about 100 μg to about 150 μg to about 260 μg to about 300 μg to about 776 μg of testosterone per day. Thus, for example, to achieve a serum blood level of about 100 μg testosterone, RELIBRA™ (applicant's trademark for gel product for women) is administered at about 0.17 g/day, which delivers about 1.7 mg/day of testosterone to the skin of which about 0.1 mg, is absorbed; or to achieve a serum blood level of about 150 μg testosterone, RELIBRA is administered at about 0.25 g/day, which delivers about 2.5 mg/day of testosterone to the skin of which about 0.15 mg, is absorbed; or to achieve a serum blood level of about 259 μg testosterone, RELIBRA is administered at about 0.44 g/day, which delivers 4.4 mg/day of testosterone to the skin of which about 0.259 mg, is absorbed; or to achieve a serum blood level of about 300 μg testosterone, RELIBRA is administered at about 0.5 g/day, which delivers 5 mg/day of testosterone to the skin of which about 0.3 mg, is absorbed; or to achieve a serum blood level of about 150 μg testosterone, RELIBRA is administered at about 0.25 g/day, which delivers about 2.5 mg/day of testosterone to the skin of which about 0.15 mg, is absorbed; or to achieve a serum blood level of about 776 μg testosterone, RELIBRA is administered at about 1.32 g/day, which delivers 13.2 mg/day of testosterone to the skin of which about 0.776 mg, is absorbed.

In one embodiment, the present invention employing testosterone is used in conjunction with a pharmacologically effective amount of an estrogenic hormone, for example, estradiol.

A class of estrogenic hormones useful in the methods, kits, combinations, and compositions of the present invention include a number of compounds that are chemical alterations produced from natural estrogens to increase their therapeutic effectiveness when administered orally. These include the steroids ethinyl estradiol, mestranol and quinestrol. In addition to these steroidal estrogens, a variety of nonsteroidal compounds having estrogenic activity have been synthesized and are used clinically. These include diethylstilbestrol, chlorotrianisene and methallenestril. The average replacement doses for several commonly used estrogens is set forth below in Table 6.

TABLE 6
Average Replacement Doses for Commonly Used Estrogens

| Estrogen | Average Replacement Dose | |
|---|---|---|
| ethinyl estradiol | 0.005-0.02 | mg/d |
| micronized estradiol | 1-2 | mg/d |
| estradiol cypionate | 2-5 | mg every 3-4 weeks |
| estradiol valerate | 2-20 | mg every other week |
| estropipate | 1.25-2.5 | mg/d |
| conjugated, esterified, or | oral 0.3-1.25 | mg/d |
| mixed estrogenic | | |
| substances | injectable 0.2-2 | mg/d |
| topical transdermal patch | 0.1-0.5 | mg/d |
| diethylstilbestrol | | |
| quinestrol | 0.1-0.2 | mg/week |
| dienestrol chlorotrianisene | 12-25 | mg/d |
| methallenestril | 3-9 | mg/d |

Oral administration of estrogens often results in adverse hepatic effects. These hepatic effects can be minimized by routes of administration that avoid first-pass hepatic exposure, such as topical (vaginal) or transdermal administration, as provided by the present invention.

Decreased production of testosterone by a woman can be caused by several factors, including, but not limited to, use of oral contraceptives; surgery, for example, removal of the uterus (hysterectomy), or removal of one of both ovaries (oophorecty/ovariectomy); estrogen replacement therapy in postmenopausal women; premature ovarian failure; adrenal dysfunction, for example primary adrenal insufficiency; corticosteroid-induced adrenal suppression; panhypopituitarism; and chronic illness, such as systemic lupus erythematosis, rheumatoid arthritis, human immunodeficiency virus (HIV) infection, chronic obstructive lung disease, and end stage renal disease.

Physiological and psychological disorders associated with testosterone deficiency in a woman include, but are not limited to, for example, decreased libido and sexual performance, decreased bone mineral density and related markers, diminished body composition, human immunodeficiency virus wasting syndrome, decreased cognition, diminished mood and self-esteem, decreased muscle mass and performance, premenstrual syndrome, central nervous system disorders, and autoimmune disease.

While not wishing to be bound by theory, it is believed that multifaceted roles of androgens as neurohormones are reflective of the widespread distribution of specific receptors in the brain. Areas where such receptors have been located include the cortex, pituitary, hypothalamus, preoptic region, and thalamus, amygdala, and brain stem. Androgen receptors not only coexist with estrogens and progesterone receptors, but also are found in regions where this is not the case. Effects of androgens are mediated via receptors, as well as through the aromatization of testosterone to estradiol by the enzyme aromatase, leading to estrogen mediated actions.

There exist well-defined patient populations where testosterone production is clearly deficient and where associated symptomatology has been described, and such populations are contemplated as falling within the scope of the present invention. These include populations include those associated with specific etiological factors, including, for example, ovarian (chemotherapy, radiation therapy, oophorectomy), adrenal (adrenal insufficiency, adrenalectomy), hypothalamic-pituitary (hypopituitarism), drug-related (corticosteroids, antiandrogenic agents, oral contraceptives, oral estrogen replacement therapies), and/or idiopathic. (See, Braunstein, G., et al., Fertility and Sterility, Vol. 77, No. 4, April 2002, pp 660-665).

Patients to be treated with the present invention include those at risk of developing a testosterone-deficient disorder, or patients currently experiencing a testosterone-deficient disorder event. Standard testosterone-deficient disorder risk factors are known to the average physician practicing in the relevant field of medicine. Patients who are identified as having one or more risk factors known in the art to be at risk of developing a testosterone-deficient disorder, as well as people who already have a testosterone-deficient disorder, are intended to be included within the group of people considered to be at risk for having a testosterone-deficient disorder event.

In addition, contemplated methods, kits, combinations, and compositions of the present invention are useful to treat testosterone deficiency in a woman, which includes a woman where testosterone production is deficient, or where the associated symptomatology related to deficient testosterone production is clinically evident. This includes, for example, a oophorectomized/hysterectomized woman, a postmenopausal woman on estrogen replacement therapy, a woman on oral contraceptives, a woman with an ovariectomy, a woman with premature ovarian failure, a woman with adrenal dysfunction, a woman with corticosteroid-induced adrenal suppression, a woman with panhypopituitarism, a woman with primary adrenal insufficiency, and a woman experiencing chronic illness, such as systemic lupus erythematosis, rheumatoid arthritis, human immunodeficiency virus (HIV) infection, chronic obstructive lung disease, and end stage renal disease.

In one embodiment of the present invention, the methods, kits, combinations, and composition are useful in treating a woman who have undergone surgery, including, for example, bilateral oophorectomy with hysterectomy, and particularly a woman whose surgery was performed at a younger age, prior to her natural menopause. In the U.S. alone, more than 250,000 women undergo combined oophorectomy/hysterectomy procedures annually and are clearly deficient in testosterone production. Serum testosterone levels typically decrease by 50% in a oophorectomized woman compared to their pre-operative levels, however, in some cases the levels may still remain within the normal reference range (approximately 20-80 ng/dL). Estrogen and progesterone levels, which are primarily dependent on ovarian secretion, are also markedly reduced after oophorectomy. The resulting multiple hormone deficiency state is associated with vasomotor symptoms, high-turnover osteoporosis, and female sexual dysfunction. While estrogen replacement therapy is standard for the treatment of vasomotor symptoms and osteoporosis in the oophorectomized/hysterectomized female, concomitant testosterone therapy has not been indicated for treatment of female sexual dysfunction or for its effects with estrogen replacement therapy on bone metabolism. Such women are contemplated as falling within the scope of the present invention.

In another embodiment of the present invention, the methods, kits, combinations, and composition are useful in treating a postmenopausal woman. In contrast to the oophorectomized state, the postmenopausal ovary may continue to synthesize testosterones in the stromal tissue at rates that are not necessarily lower than the premenopausal period. In some postmenopausal women, testosterone levels increase as a consequence of the stromal response to elevated luteinizing hormone levels, while in others testosterone levels decrease or remain the same. Since estrogen replacement therapy lowers luteinizing hormone levels, ovarian testosterone secretion would be expected to decrease in postmenopausal women who receive estrogen replacement therapy. With oral estrogen replacement therapy preparations, the fall in testosterone levels may be obscured by the concomitant rise in sex hormone binding globulin levels, which reduces testosterone clearance. However, free and/or bioavailable testosterone levels are found to be lower in a postmenopausal woman receiving oral estrogen replacement therapy. While the effects of transdermal estrogen replacement therapy on the androgen/luteinizing hormone status of postmenopausal women has not been studied, a reduction in total and free testosterone levels, associated with a decrease in luteinizing hormone levels, would also be expected. As many postmenopausal women experience symptoms of female sexual dysfunction that are not ameliorated by estrogen replacement therapy, it is believed that testosterone deficiency is a contributing factor, and this group of women would fall within the scope of the present invention.

In yet another embodiment of the present invention, the methods, kits, combinations, and composition are useful in treating a woman who uses oral contraception. Oral contraception is the most common method of contraception among adolescents, and overall about 46% of the sexually active population use oral contraception. The most common type of oral contraceptive contains both estrogen and progestin and has proven to be about 99% effective. Thus, almost half of all premenopausal women (<44 years old) are potentially taking oral contraceptives. In comparison to healthy “cycling” women, the testosterone levels in women treated with estrogen-containing oral contraceptives are markedly lower, particularly when compared at the pre-ovulatory phase of the normal cycle, when testosterone levels are highest. This effect result from the luteinizing hormone suppression produced by oral contraceptives and is analogous to the effect of estrogen replacement therapy described above. Also oral contraceptive use generally increases sex hormone binding globulin concentration in women leading to increase binding of testosterone resulting in a decrease level of free testosterone. Psychosexual aspects of perception are affected by the lower testosterone levels and may be related to the clinical observation of decreased libido in some women using oral contraceptives.

In yet another embodiment of the present invention, the methods, kits, combinations, and composition are useful in treating a woman who have an undergone an ovariectomy by, for example, surgery, chemical means, irradiation, or gonadotropin-releasing hormone antagonists. Such surgery leads to decreased ovarian androgen product.

In another embodiment of the present invention, the methods, kits, combinations, and composition are useful in treating a woman with premature ovarian failure. Premature ovarian failure, such as that associated with Turner's Syndrome or the autoimmune or idiopathic destruction of the ovary, is associated with impaired testosterone production.

In still another embodiment of the present invention, the methods, kits, combinations, and composition are useful in treating a woman who has decreased adrenal function. Decrease adrenal function, which may result from a variety of causes, represents another category of patients where testosterone production may be reduced by approximately 50%. Primary adrenocortical deficiency, or Addison's disease, is a rare endocrine disorder with multiple etiologies, including tuberculosis and fungal infections. The estimated prevalence in women is approximately 5 per 100,000. Due to the lack of gluco- and mineral corticoid secretion, Addison's disease can be life threatening. While some researchers have noted the associated testosterone deficiency, replacement therapy is often ignored. As the adrenocorticotropic hormone appears to be the primary stimulator of adrenal androgen production, deficient adrenocorticotropic hormone secretion can also lead to testosterone deficiency in women. This can result from pituitary disease or surgery, for example, secondary adrenocortical deficiency, or as a pharmacological effect of exogenous corticosteroid administration that can suppress adrenocorticotropic hormone secretion.

In one embodiment of the present invention, the methods, kits, combinations, and composition are useful in treating a woman where chronic corticosteroid therapy is administered. Chronic corticosteroid therapy is used for a variety of conditions, which include rheumatoid arthritis, systemic lupus erythematosus, Sjogren's syndrome, immunosuppression for transplants, asthma, etc. Corticosteroid-induced adrenal suppression may thus represent the largest group of patients with deficient adrenal androgen production. Androgen deficiency is recognized as a contributory factor to corticosteroid-induced osteoporosis. By stimulating bone formation (osteoblast activity), testosterone replacement is beneficial in the treatment of corticosteroid-induced osteoporosis in premenopausal women, and is beneficial in estrogen replacement therapy when treating postmenopausal women. In a woman with autoimmune disorders, such as rheumatoid arthritis and systemic lupus erythematosus, testosterone deficiency can contribute to the underlying tendency to produce autoantibodies, as has been seen in a variety of animal models of autoimmune disease. Testosterone replacement can thus help to ameliorate the autoimmune disease process, itself. Despite these considerations, the potential therapeutic benefits of testosterone replacement in treating corticosteroid suppressed women have largely been ignored.

In another embodiment of the present invention, the methods, kits, combinations, and composition are useful in treating a panhypopituitarism woman. Panhypopituitarism from any cause is attended by a severe testosterone deficiency because of derangement of androgen secretion by both the ovaries and the adrenal glands.

In yet another embodiment of the present invention, the methods, kits, combinations, and composition are useful in treating a woman with primary adrenal insufficiency. Primary adrenal insufficiency is associated with testosterone deficiency.

In one embodiment of the present invention, the methods, kits, combinations, and composition are useful in treating a woman with chronic illnesses. Chronic illnesses in a woman are attended by decreased circulating testosterone concentrations. Glucocorticoid administration inhibits adrenal androgen production by their inhibitory effects on adrenocorticotropic hormone secretion. In addition, glucocorticoids also have inhibitory effects at all levels of the hypothalamic-pituitary-ovarian axis.

In still another embodiment of the present invention, the methods, kits, combinations, and composition are useful in treating a human immunodeficiency virus-positive woman. In contrast to human immunodeficiency virus-positive men, where testosterone deficiency is common, it is not known whether human immunodeficiency virus-positive women are deficient in testosterone. Amenorrhea, which appears to be increased in women with acquired immunodeficiency syndrome (AIDS), may be an indication that ovarian steroid production is diminished. Adrenal function can also be deficient in acquired immunodeficiency syndrome patients due to cytomegalovirus infection, tuberculosis and/or fungal infections. Megestrol acetate, a progestational agent used to stimulate appetite in human immunodeficiency virus infected persons, suppresses gonadotropins and is it believed to lower testosterone levels in women, similar to its effects in men. In addition, the use of oral contraceptives by a human immunodeficiency virus-positive woman also reduces testosterone levels, as described above in normal women. Physiological testosterone replacement can be used as an anabolic agent for treating/preventing the wasting syndrome and for enhancing quality of life in a woman.

The methods, kits, combinations, and compositions of the present invention are also useful to treat a number of physiological and psychological parameters associated with testosterone deficiency in a woman, and include, for example, increasing libido and improving sexual performance and dysfunction, increasing bone mineral density and related markers, improving body composition, preventing human immunodeficiency virus wasting syndrome, improving cognition, improving mood and self-esteem, improving muscle mass and performance, treating premenstrual syndrome, treating central nervous system disorder, and treating autoimmune diseases.

In one embodiment of the present invention, the methods, kits, combinations, and composition are useful in treating the libido of a woman. Testosterone concentrations clearly affect female libido. Over the past few decades, several correlational studies found that higher testosterone levels were associated with less sexual avoidance, more sexual gratification, more sexual thoughts, more initiation of sexual activity, higher levels of sexual interest and desire, and more anticipation of sexual activity. More recently, found a correlation between sexual desire and testosterone in a subset of women, those who were human immunodeficiency virus-positive.

In one embodiment of the present invention, the methods, kits, combinations, and composition are useful in treating sexual performance in a woman. Studies have shown that testosterone influences sexual performance in women. Correlational studies have found that testosterone is associated with higher sexual arousability as measured by vasocongestive responses to erotic films, increased frequency of masturbation, increased frequency of coitus, and a higher number of sexual partners. Another correlational study also showed that testosterone is associated with decreased vaginal atrophy.

In another embodiment of the present invention, the methods, kits, combinations, and composition are useful in treating female sexual dysfunction in a woman. Surgical menopause, that is, total abdominal hysterectomy and bilateral salpingo-oophorectomy, performed prior to the natural menopause causes a syndrome of female sexual dysfunction in a significant number of women that is unrelieved by conventional estrogen replacement therapy. The sexual components of this syndrome include decreased libido, decreased arousal and a diminished ability to attain orgasm. The psychological components include decreased energy, depressed mood, and a general decrease in well-being. These are generally distinguishable from the classic estrogen deficiency symptoms of vaginal atrophy, diminished lubrication, hot flushes and emotional liability that can adversely affect sexual function and psychological well-being in menopausal women who do not receive adequate estrogen replacement therapy. Rather than estrogen deficiency, the hormonal basis for this syndrome is attributed to a testosterone deficiency state resulting from the absent ovarian production of testosterone and its precursors.

In one study, the effects of testosterone in women with impaired sexual function after surgically induced menopause were evaluated using a transdermal patch. Seventy-five women, 31 to 56 years old, who had undergone oophorectomy and hysterectomy received conjugated equine estrogens (at least 0.625 mg per day orally) and, in random order, 150 μl of testosterone, and 300 μg of testosterone per day transdermally for 12 weeks each. Outcome measures included scores on the Brief Index of Sexual Functioning for Women (BISF), the Psychological Well-Being Index (PGWI), and a sexual function diary completed over the telephone. The mean (±SD) serum free testosterone concentration increased from 1.2±0.8 pg/mL during placebo treatment to 3.9±2.4 pg/mL and 4.9±4.8 pg/mL during treatment with 160 and 300 μg of testosterone per day, respectively (normal range, 1.3 to 6.8 pg/mL. Despite an appreciable placebo response, the higher testosterone dose resulted in further increases in scores for frequency of sexual activity and pleasure-orgasm in the Brief Index of Sexual Functioning for Women (P=0.03 for both comparisons with placebo). At the higher dose, the percentages of women who had sexual fantasies, masturbated, or engaged in sexual intercourse at least once a week increased two to three times from base line. The positive-well-being, depressed-mood, and composite scores of the Psychological Well-Being Index also improved at the higher dose (P=0.04, P=0.04, respectively, for the comparison with placebo), but the scores on the telephone-based diary did not increase significantly.

In another embodiment of the present invention, testosterone therapy is used in conjunction with estrogen therapy. Studies have shown that testosterone and estrogen replacement resulted in increased sexual desire, frequency of sexual fantasies, sexual arousal, and coital or orgasmic frequency compared to those given estrogen alone or a placebo reported that women receiving estrogen plus testosterone experienced more increased libido, activity, satisfaction, pleasure, fantasy, orgasm, and relevancy as compared to women receiving estrogen alone. Treatment with Premarin and methyltestosterone resulted in significantly increased reports of pleasure from masturbation. Treatment with estrogen and methyltestosterone similarly results in increased sexual interest. Most recently, it has been found that transdermal testosterone treatment in women after oophorectomy improved sexual function and psychological well-being. It is contemplated that testosterone administration alone will have therapeutic benefits if given without estrogen. For example, women with hypothalamic amenorrhea show increased vaginal vasocongestion with testosterone treatment compared to a placebo.

In still another embodiment of the present invention, the methods, kits, combinations, and composition are useful in treating decreased bone density in a woman. Another physiologic parameter linked to testosterone administration in women is decreased bone mineral density. Several correlational studies have shown that increased testosterone concentrations are associated with increased bone mineral density. It has been found that higher bioavailable testosterone levels were associated with higher bone mineral density in the ultradistal radius in women. Women having polycystic ovary syndrome had neck bone mineral density positively correlated to free testosterone levels. Upper body bone mineral density had significant correlation with testosterone. A cross-sectional analysis of sex hormone concentrations and bone mineral density in women recruited for a prospective study of risk factors for osteoporosis and found a significant positive correlation between testosterone and bone mineral density. Another study involved an age-stratified sample of 304 women and found a correlation coefficient between bone mineral density and testosterone as shown below in Table 7:

TABLE 7
Correlational Coefficients between
Testosterone and Bone Mineral Density*

| | Total | Bioavailable |
|---|---|---|
| | Testosterone | Testosterone |
| Total body | 0.22 | 0.22 |
| Lateral spine | 0.27 | 0.29 |
| Proximal femur | 0.25 | 0.30 |
| Radius | 0.27 | 0.28 |

*Khosla S. et al., J Clin Endocrinol Metab. .1998 Jul;83(7): 2266-74.

As with libido and sexual performance, testosterone is often given in conjunction with estrogen in order to prevent bone loss or increase bone mineral density. For example, in a cross sectional study, it was found that subcutaneous estradiol (75 mg) and testosterone (100 mg) prevented osteoporosis and maintained normal bone mineral density in postmenopausal women. In another study the effects of estrogen given alone to those of estrogen plus androgen therapy in postmenopausal women. While the estrogen-only group had a reduction in serum markers of bone formation, women treated with combined estrogen and testosterone had increased bone formation markers. Similarly, it has been shown that estrogen and testosterone replacement with implant pellets increases bone mass more than estrogen implants alone, increased bone mineral density by 5.7% in the spine and 5.2% in the neck femur region. Treatment with estrogen and methyltestosterone similarly results in increased spine and hip bone mineral density. Also, it has been reported that orally given estrogens and methyltestosterone prevented bone loss and increased bone mineral density in the spine and hip.

In another embodiment of the present invention, the methods, kits, combinations, and composition are useful in treating body composition of a woman. Testosterone has been linked to improved body composition in women. Testosterone is positively correlated to body mass index and exogenous androgens influenced body composition and regional body fat distribution in obese postmenopausal women. Other researchers have found an increase in fat-free mass and a reduced fat mass to fat free mass ratio in postmenopausal women treated with concurrent estrogen-testosterone therapy. Thus, administration of testosterone to normal women or those having testosterone deficiencies may have a therapeutic improvement in body composition.

In still another embodiment of the present invention, the methods, kits, combinations, and composition are useful in treating or preventing human immunodeficiency virus wasting syndrome in a woman. In recent years, researchers have found that testosterone administration to women infected with human immunodeficiency virus may treat or prevent human immunodeficiency virus wasting syndrome. It has been found that lower free testosterone levels in human immunodeficiency virus-infected women using a tracer analog method. For example, testosterone replacement in a patch delivering 150 ug/day of testosterone to human immunodeficiency virus-infected women had a 4% increase in body weight over 12 weeks. In addition, the patients had an improved quality of life. Thus, testosterone administration can be used as a method of preventing wasting in women suffering from acquired immunodeficiency syndrome or related disorders.

In yet another embodiment of the present invention, the methods, kits, combinations, and composition are useful in treating or preventing short-term and long-term memory and other higher-order cognitive functions in a woman, including those caused by central nervous disorders, for example. Sex steroids are important for short-term and long-term memory and other higher-order cognitive functions. Postmenopausal women receiving estrogen plus testosterone following oophorectomy had higher scores on two tests of short-term memory, a test of long-term memory, and a test of logical reasoning. It has been reported that the administration of testosterone is associated with better visio-spacial function and verbal skills. Women with high testosterone levels scored higher on special/mathematical tasks than women with low testosterone concentrations. Women with higher Mini-Mental State Examination scores had significantly higher mean total and bioavailable testosterone concentrations. Testosterone levels are also related to verbal fluency. Again, the benefits of testosterone administration on cognitive parameters may be optimized by concurrent estrogen administration. For example, subcutaneous implants of oestradiol (40 mg) and testosterone (100 mg) have shown increases in concentration.

In one embodiment of the present invention, the methods, kits, combinations, and compositions are useful in treating or preventing a mood or self-esteem disorder in a woman. Parameters associated with testosterone serum levels in women are mood and self-esteem. Menopausal women who received both estrogen and testosterone felt more composed, elated, and energetic than those who were given estrogen alone. Similarly, testosterone concentrations are positively correlated to self-esteem. Thus, it is contemplated that testosterone therapy will improve mood when used alone or in conjunction with estrogen.

In another embodiment of the present invention, the methods, kits, combinations, and composition are useful in increasing muscle size and performance in a woman. Androgens and anabolic steroids have long since been used to increase muscle size and performance in men. Researchers have recently also found that testosterone is an important determinant of greater muscle size in women with polycystic ovary syndrome. Thus, administration of testosterone to a normal or testosterone deficient woman may be useful for improving muscle mass and performance.

Many of the symptoms described above fall under the umbrella of what is commonly considered to be premenstrual syndrome (PMS). In general, lower levels of testosterone throughout the menstrual cycle have been reported in women who suffer from premenstrual syndrome compared with controls. Testosterone replacement is currently used as a management of premenstrual syndrome in the United Kingdom and Australia. Managing premenstrual syndrome with oestradiol/testosterone implants resulted in improvements in libido, enjoyment of sex, and tiredness. Thus, it is contemplated that the methods, kits, combinations, and compositions of the present invention can be useful in treating premenstrual syndrome in a woman, especially in conjunction with estrogen administration.

In one embodiment of the present invention, the methods, kits, combinations, and composition are useful in suppressing both cell-mediated and humoral immune responses in a woman. Androgens appear to suppress both cell-mediated and humoral immune responses. Many researchers have advocated increasing testosterone levels in women as protective against autoimmune disease, such as rheumatoid arthritis. Testosterone administration therefore is contemplated to be effective in treating a woman with such disorders.

Toxicity and therapeutic efficacy of the therapeutic agents of the present invention can be determined by standard pharmaceutical procedures, for example, for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The active agents of the present invention may be administered, if desired, in the form of a salt, an ester, a hydrate, an amide, an enantiomer, an isomer, a tautomer, a polymorph, a prodrug, a derivative and the like, provided the salt, ester, hydrate, amide, enantiomer, isomer, tautomer, polymorph, prodrug, or derivative is suitable pharmacologically, that is, effective in the present methods, kits, combinations, and compositions. Salts, esters, hydrates, amides, enantiomers, isomers, tautomers, polymorphs, prodrugs, or derivatives of the active agents may be prepared using standard procedures known to those skilled in the art of synthetic organic chemistry and described, for example, by J. March, Advanced Organic Chemistry; Reactions, Mechanisms and Structure, 4th Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, for example, hydrochloric acid, hydrobromic acid, sulfuric acid, nitric acid, phosphoric acid, and the like. An acid addition salt may be reconverted to the free base by treatment with a suitable base. Illustratively, acid addition salts of the active agents herein include halide salts, such as may be prepared using hydrochloric or hydrobromic acids. Illustratively, basic salts include alkali metal salts, for example, the sodium salt, and copper salts. Preparation of esters involves functionalization of hydroxyl and/or carboxyl groups which may be present within the molecular structure of the drug. The esters are typically acyl-substituted derivatives of free alcohol groups, that is, moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

The therapeutic agents of the present invention can be formulated as a single pharmaceutical composition containing at least one therapeutic agent, or as independent multiple pharmaceutical compositions where each composition contains at least one therapeutic agent. Pharmaceutical compositions according to the present invention include those compositions with at least one therapeutic agent formulated for percutaneous administration. Percutaneous administration includes transdermal delivery systems that include patches, gels, tapes and creams, and can contain excipients such as alcohols, penetration enhancers, and thickeners, as well as solubilizers (for example propylene glycol, bile salts, and amino acids), hydrophilic polymers (for example, polycarbophil and polyvinylpyrolidone), and adhesives and tackifiers (for example, polyisobutylenes, silicone-based adhesives, acrylates and polybutene).

The therapeutic agents of the present invention can then be administered percutaneously in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

The compositions of the present invention can be administered for treating, preventing, or reducing the risk of developing a testosterone deficiency in a mammal by any means that produce contact of these compounds with their site of action in the body, for example in the ileum, the plasma, the central nervous system or the liver of a mammal.

Additionally, the methods, kits, combinations, and compositions of the present invention may optionally include salts, emollients, stabilizers, antimicrobials, fragrances, and propellants.

In another embodiment of the present invention, the therapeutic agents come in the form of kits or packages containing testosterone. Illustratively, the kits or packages contain testosterone in a dosage form suitable for percutaneous administration, for example, a gel or a patch, in amounts for the proper dosing of the drugs. The therapeutic agents of the present invention can be packaged in the form of kits or packages in which the daily (or other periodic) dosages are arranged for proper sequential or simultaneous administration. The present invention further provides a kit or package containing a plurality of dosage units, adapted for successive daily administration, each dosage unit comprising at least one of the therapeutic agents of the present invention. This drug delivery system can be used to facilitate administering any of the various embodiments of the therapeutic compositions. In one embodiment, the system contains a plurality of dosages to be to be administered daily or weekly via percutaneous administration. The kits or packages also contain a set of instructions for the patient.

The present methods, kits, combinations, and compositions can also be used in “combination therapy” with another steroid such as, for example, progesterone, or a pharmaceutical agent that increases testosterone levels in a mammal, such as, for example, an agent that inhibits the synthesis of the sex hormone binding globulin, or, as mentioned above, with an estrogenic hormone.

In another embodiment, the methods, kits, combinations, and compositions are used in conjunction with other steroids or pharmaceutical agents effective at treating, preventing, or reducing the risk of developing a testosterone-deficient disorder in a subject.

A class of steroids or pharmaceutical agents that increases testosterone levels in a mammal useful in the methods, kits, combinations, and compositions of the present invention include compounds that inhibit the synthesis of sex hormone binding globulin. Sex hormone binding globulin is a serum protein, and is known to bind to testosterone and estradiol, effecting the biological activity of these hormones. Specific compounds of interest that inhibit the synthesis the sex hormone binding globulin include but are not limited to methyltestosterone and fluoxymesterone, and all salts, esters, hydrates, amides, enantiomers, isomers, tautomers, polymorphs, prodrugs, or derivatives of these compounds. Methyltestosterone is currently available in various formulations including those available orally, for example ANDROID® and TESTRED®. Fluoxymesterone is also currently available in various formulations including those available orally, for example HALOSTESTIN®. Combinations of the above mentioned compounds can be used.

While not wishing to be bound by theory, it is believed that methyltestosterone decreases hepatic synthesis of endogenous proteins like sex hormone binding globulin. This decrease in synthesis produces a decline in blood concentrations of sex hormone binding globulin, which is the primary means of endogenous hormone transport. The decrease in sex hormone binding globulin subsequently causes an increase in free-hormone concentration for binding at the receptor. Transdermal application of an androgen, for example, testosterone, or an estrogen, for example, estradiol, bypasses first-pass metabolism and can provide a means of increasing hormone concentrations in the bloodstream. Thus, when used in combination, methyltestosterone and percutaneously administered testosterone (and optionally estradiol) produce a greater therapeutic effect and provide a means of increasing hormone concentrations in the bloodstream. Methyltestosterone and testosterone (and optionally estradiol) produce a greater therapeutic effect than either entity alone because the decrease in hormone binding ability is coupled with an increased hormone bioavailability, producing higher free-hormone concentrations that would be produced by testosterone alone.

In another embodiment of the present invention, the estrogenic hormone that can be used in conjunction with the methods, kits, combinations, and composition is the naturally occurring estrogen 17 beta-estradiol (beta-estradiol; 1,3,5(10)-estratriene-3,17 beta-diol). Other estrogenic steroid hormones can be used in partial or complete replacement of 17 beta-estradiol, for example, an ester which is biologically compatible and can be absorbed effectively transdermally. The estradiol esters can be, illustratively estradiol-3,17-diacetate; estradiol-3-acetate; estradiol-17-acetate; estradiol-3,17-divalerate; estradiol-3-valerate; estradiol-17-valerate; 3-mono, 17-mono and 3,17-dipropionate esters, corresponding cypionate, heptanoate, benzoate and the like esters; ethynil estradiol; estrone and other estrogenic steroids and salts, enantiomers, isomers, tautomers, prodrugs and derivatives thereof that are possible to administer by transdermal route. Other estrogen-related compounds that may be used in the methods, kits, combinations, and compositions of the present invention include, but are not limited to conjugated estrogens (including estrone sulfate, equilin, and 17-.alpha.-dihydroequilin), estradiol valerate, estriol, estrone, estrone sulfate, estropipate, ethinyl estradiol, mestranol, and all salts, esters, hydrates, amides, enantiomers, isomers, tautomers, polymorphs, prodrugs, or derivatives of these compounds.

Estrogenic hormones are currently available in various formulations including, but not limited to those available as a cream, pessary, vaginal ring, vaginal tablet, transdermal preparation, gel, and oral tablet. Examples of vaginal creams include PREMARIN® (conjugated estrogen), ORTHO DIENOSTEROL® (dienosterol), and OVESTIN® (estriol). Available pessary formulations include ORTHO-GYNEST® (estriol), and TAMPOVAGAN® (stilbestrol). An example of a vaginal ring formulation is ESTRING® (estradiol), and an example of a vaginal tablet is VAGIFEM® (estradiol). Available transdermal estrogen preparations containing estradiol include ERC ALORA®, CLIMARA®, DERMESTRIL®, ESTRADERM®, ESTRADERM® TTS, ESTRADERM® MX, EVOREL®, FEMATRIX®, FEMPATCH®, FEMSEVEN®, MENOREST®, PROGYNOVA® TS, and VIVELLE®. Estrogen gels containing estradiol include ESTROGEL (under development by Applicant), and SANDRENA®. Estradiol is also available formulated as an implant pellet, for example, ESTRADIOL IMPLANT®. Tablet formulations include PREMARIN® (conjugated estrogen), ESTRATAB® (esterified estrogen), ESTRATEST® (esterified estrogen, methyltestosterone), MENEST® (esterified estrogen), CLIMAGEST®, (estradiol), CLIMAVAL® (estradiol), ELLESTE SOLO® (estradiol), ESTRACE® (estradiol), PROGYNOVA® (estradiol), ZUMENON® (estradiol), HORMONIN® (estradiol, estrone, estriol), HARMOEN® (estrone), OGEN® (estropipate), and ORTHO-EST® (estropipate); and all salts, esters, hydrates, amides, enantiomers, isomers, tautomers, polymorphs, prodrugs, or derivatives of these compounds.

Combinations of the above mentioned estrogenic hormones can be used. In one embodiment of the present invention, the serum blood level of estrogen is raised to at least about 60 pg estrogen/ml blood serum within 24 hours after a single administration of a dosage unit of the present invention containing estrogen.

In one embodiment, the estrogenic hormone is formulated for percutaneous administration in a hydroalcoholic gel. The gel comprises one or more lower alcohols, a penetration enhancing agent, a thickener, and water. Additionally, the estrogenic gel optionally includes salts, emollients, stabilizers, antimicrobials, fragrances, and propellants.

Illustratively, the estrogenic gel is comprised of the following substances as shown below in Table 8, in approximate amounts.

TABLE 8
Composition of ESTROGEL

| | AMOUNT | |
|---|---|---|
| | (w/w) PER | |
| SUBSTANCE | 100 g OF GEL | |
| 17-beta-oestradiol | 0.06 | g |
| Carbopol 980 | 1 | g |
| Triethanolamine | 1.35 | g |
| Ethanol (95% v/v) | (59 | ml) |
| Purified water (qsf) | 100 | g |

One skilled in the art will appreciate that the constituents of this formulation may be varied in amounts yet continue to be within the spirit and scope of the present invention. For example, the composition may contain about 0.1 to about 10 g of estradiol, about 0.1 to about 5 g CARBOPOL, about 0.1 to about 5 g triethanolamine, and about 30 to about 98 g ethanol.

The phrase “combination therapy” embraces the administration of a steroid in the testosterone synthesis pathway in conjunction with another steroid or pharmaceutical agent that increases testosterone levels in a mammal, or with an estrogenic hormone, as part of a specific treatment regimen intended to provide a beneficial effect from the co-action of these therapeutic agents for the treatment of a testosterone-deficient disorder in a mammal. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single gel having a fixed ratio of each therapeutic agent or in multiple, single capsules, tablets, or gels for each of the therapeutic agents. Sequential or substantially simultaneous administration of each therapeutic agent can be effected by any appropriate route including, but not limited to, oral routes, percutaneous routes, intravenous routes, intramuscular routes, and direct absorption through mucous membrane tissues. The therapeutic agents can be administered by the same route or by different routes. For example, a first therapeutic agent of the combination selected may be administered orally, while the other therapeutic agents of the combination may be administered percutaneously. Alternatively, for example, all therapeutic agents may be administered percutaneously, or one of the therapeutic agents may be administered intravenously, intramuscularly, or by direct absorption through mucous membrane tissues. The sequence in which the therapeutic agents are administered is not narrowly critical. “Combination therapy” also can embrace the administration of the therapeutic agents as described above in further combination with other biologically active ingredients, such as, but not limited to, agents for improving sexual performance, and non-drug therapies, such as, but not limited to, surgery.

An agent for improving sexual performance that can be used in conjunction with the methods, kits, combinations, and composition of the present invention include, for example, ArginMax™; SureGasm™; Vitara™; Viacreme™; Niagara™; Ultimate Libido™; X-cite™; alprostadil; bupropion (Wellutrin™ SR, Zyban™, Zyban™ LP, Quomen™, Zyntabac™); REC-2615 (Recordati); phentolamine (Bimexes™, Zonagen™, Erxin™) (GlaxoSmithKline, Zonagen); HMP-12 (HMP-1991012) (Molecular Design); LibiGel™-E/T (Antares Pharma); nicotinic acid; 3-pyridinecarboxylic acid; androst-4-en-one, 17-hydroxy-(17beta); P2Y2 agonist; TA-179 (Tanabe Seiyaku); stearyl-norleucine-VIP (SNV) (Senetek); NMI-870, NMI-921 (NitroMed); PT-141 (Palatin Technologies, U.S. Pat. No. 6,051,555); alprostadil (LAM Pharmaceutical) (U.S. Pat. Nos. 6,224,573; 6,306,841; 5,942,545; 6,046,244); IPM tech (LAM Pharmaceutical); Ramot project No. 981 (Ramot); apomorphine HCl (Nastech, U.S. Pat. No. 6,436,950); LGD-2226, LG-121071 (Ligand); vardenafil (Bay-38-9456, Nuviva, Levitra (Bayer)); PT-14 (Palatin Technologies); flibanserin (Boehringer Ingelheim) (WO 94/24125); phentolamine mesylate (Vasomax™, Z-Max™, Vasofem™, Regitin™) (Zonagen) (U.S. Pat. No. 5,731,339); 7-day HRT transdermal (EMD-61409, Fem7™, FemSeven™) (Merck); Iosatan+HCTZ (Pinzaar™ plus, Hizaar™, Lortann™, Neo-Lotan™) (Bristol-Meyer Squibb) (WO 94/09778); MS-325 (AngioMark™, Vasovist™, ZK-236018) (EPIX Medical) (WO 06/23526); tadalafil (Cialis™, PDE-A, PDE-CV, GF-196960, IC-351) (Eli Lilly) (U.S. Pat. No. 6,143,746; WO 95/19978); atipomezole (Antisedan™, MPV-1248) (Orion Pharama) (EP 183492); duloxetine HCl (Eli Lilly) (U.S. Pat. No. 5,362,886); bupropion (323U66, amfebutamone, BW-323U, Wellbutrin™) (GlaxoSmithKline) (U.S. Pat. No. 4,424,363; an agent that causes vasodilatation (see, for example, U.S. Pat. Nos. 5,877,216; 6,506,765; 6,395,74; and 6,294,550); an agent that donates, transfers or releases nitrogen monoxide (see, for example, U.S. Pat. No. 6,472,425); a 5-HT1A receptor agonist (for example, VML-670, CEB-1555 (Eli Lilly)); agent that induces the production of endogenous endothelium-derived relaxing factor; an agent that stimulates endogenous synthesis of nitrogen monoxide; an agent that is a substrate for nitric oxide synthase; or a phosphodiesterase inhibitor (see, for example, U.S. Pat. No. 6,469,016).

An agent that causes vasodilatation that can be used in conjunction with the methods, kits, combinations, and composition of the present invention, include, for example, a vasodilator, ergotamine, a nitrovasodilator, an ACE inhibitor, a leukotriene inhibitor, an angiotensin receptor antagonist, a phosphodiesterase inhibitors, a direct vasodilator, an adrenergic receptor antagonist, a beta 2-adrenergic agonist, a calcium channel blocking drug, an alpha blocker, a beta blocker, a lympathomimetic, a vitamin, an organic nitrate, a serotonin receptor-blocking agent, an angina blocking agent, a anti-hypertensive agent, a cardiac stimulating agent, an agents which improve renal and vascular function, a sympathomimetic amine, a naturally occurring prostaglandin (see, for example, U.S. Pat. No. 6,036,977), a synthetic prostaglandin derivative, an endothelial-derived relaxation factor, a vasoactive intestinal polypeptide agonist, a smooth muscle relaxant, a leukotriene inhibitor. Examples of a vasodilator include apomorphine, apomorphine N-glucuronide, apomorphine O-glucuronide, apomorphine O-sulfate, apomorphine N-sulfate, norapomorphine, norapomorphine O-glucuronide, norapomorphine N-glucuronide, norapomorphine O-sulfate, norapomorphine N-sulfate, niacin, nitroglycerine, hydrochloride, hydrochlorothiazide, nilatrin hydrochloride, pentoxyphylene, phenoxybenzamine, dichlophenac, papaverine, hydralazine, sodiumnitroprusside, isoxaprine hydrochloride, epoprostenol sodium, nylidrin hydrochloride, tolazoline hydrochloride, nicotinyl alcohol, nicotinic acid, nicotinic acid precursors, esters of nicotinic acid and phentolamine, phentolamine mesylate, pentolamine hydrochloride, yohimbine, thymoxamine imipramine, verapamil, isoxsuprine, naftidrofuryl, tolazoline, hydroisosorbide, dibenamine, dinitrate, captopril, enalapril, enalaprilat, quinapril, lisinopril, ramipril, losartan, amrinone, milrinone, vesnarinone, nicorandil, prazosin, labetalol, celiprolol, carvedilol, bucindolol, nifedipine dobutamine, minoxidil, a nitrate, and nylidrin; or a salt, an ester, a hydrate, an amide; an enantiomer, an isomer, a tautomer, a polymorph, a prodrug, or a derivative thereof. Combinations of the above mentioned agents that cause vasodilatation can be used.

Illustratively, a nitrate that can be used in conjunction with the methods, kits, combinations, and composition of the present invention, include, nitroglycerin, isosorbide dinitrate, erythrityl tetranitrate, amyl nitrate, molsidomine, linsidomine chlorhydrate, S-nitroso-N-acetyl-d,l-penicillamine and S-nitroso-N-glutathione; or a salt, an ester, a hydrate, an amide, an enantiomer, an isomer, a tautomer, a polymorph, a prodrug, or a derivative thereof. Combinations of the above mentioned nitrate can be used.

Illustratively, an alpha-blocker that can be used in conjunction with the methods, kits, combinations, and composition of the present invention, include, phenoxybenzamine, dibenamine, doxazosin, terazosin, phentolamine, tolazoline, prazosin, trimazosin, alfuzosin, tansulosin and indoramin; ergot alkaloids such as ergotamine and ergotamine analogs, for example, acetergamine, brazergolirie, bromerguride, cianergoline, delorgotrile, disulergine, ergonovine maleate, ergotamine tartrate, etisulergine, lergotrile, Iysergide, mesulergine, metergoline, metergotamine, nicergoline, pergolide, propisergide, proterguride and terguride; antihypertensive agents such as diazoxide, hydralazine and minoxidil; nimodepine; pinacidil; cyclandelate; dipyridamole; isoxsuprine; chlorpromazine; haloperidol; yohimbine; and trazodone; or a salt, an ester, a hydrate, an amide, an enantiomer, an isomer, a tautomer, a polymorph, a prodrug, or a derivative thereof. Combinations of the above mentioned alpha-blockers can be used.

Illustratively, a naturally occurring prostaglandin that can be used in conjunction with the methods, kits, combinations, and composition of the present invention, include, prostaglandin E₀, prostaglandin E₁, prostaglandin A₁, prostaglandin B₁, prostaglandin F₁ₐₗₚₕₐ, 19-hydroxy-prostaglandin A₁, 19-hydroxy-prostaglandin B₁, prostaglandin E₂, prostaglandin A₂, prostaglandin B₂, 19-hydroxy-prostaglandin A₂, 19-hydroxy-prostaglandin B₂, prostaglandin E₃, prostaglandin F₃ₐₗₚₕₐ, prostaglandin I₂. Examples of a synthetic prostaglandin, include, carboprost tromethamine, dinoprost tromethamine, dinoprostone, lipoprost, gemeprost, metenoprost, sulprostone and tiaprost; or a salt, an ester, a hydrate, an amide, an enantiomer, an isomer, a tautomer, a polymorph, a prodrug, or a derivative thereof. Combinations of the above mentioned naturally occurring prostaglandins can be used.

An endothelial-derived relaxation factors include, for example, sodium nitroprusside, diazenium diolates, (Z)-1-{N-methyl-N-[6-(N-methyl-ammoniohexyl)amino]}diazen-1-ium-1,2-diolate, (Z)-1-[N-(3-ammoniopropyl)-N-(n-propyl)amino]diazeri-1-ium-1,2-diolate, (Z)-1-{N-[3-aminopropyl]-N-[4-(3-aminopropylammonio)butyl]amino}diazen-1-ium-1,2-diolate and sodium (Z)-1-(N,N-diethylamino)-diazen-1-ium-1,2-diolate; or a salt, an ester, a hydrate, an amide, an enantiomer, an isomer, a tautomer, a polymorph, a prodrug, or a derivative thereof. Combinations of the above mentioned endothelial-derived relaxation factors can be used.

Suitable phosphodiesterase inhibitors that can be used in conjunction with the methods, kits, combinations, and composition of the present invention, include, but are not limited to, inhibitors of type III phosphodiesterase (cAMP-specific-cGMP inhibitable form), type IV phospodiesterase (high affinity-high specificity cAMP form) and type V phosphodiesterase (the cGMP specific form). Additional inhibitors that may be used in conjunction with the present invention are cGMP-specific phosphodiesterase inhibitors other than type V inhibitors, including pyrazolopyrimidinone, for example.

Examples of type III phosphodiesterase inhibitors that can be used in conjunction with the methods, kits, combinations, and composition of the present invention, include, but are not limited to, bypyridines such as milrinone and amirinone, imidazolones such as piroximone and enoximone, dihydropyridazinones such as imazodan, 5-methyl-imazodan, indolidan and ICI118233, quinolinone compounds such as cilostamide, cilostazol and vesnarinone, and other molecules such as bemoradan, anergrelide, siguazodan, trequinsin, pimobendan, SKF-94120, SKF-95654, lixazinone and isomazole; or a salt, an ester, a hydrate, an amide, an enantiomer, an isomer, a tautomer, a polymorph, a prodrug, or a derivative thereof. Combinations of the above mentioned type III phosphodiesterase inhibitors can be used.

Examples of type IV phosphodiesterase inhibitors that can be used in conjunction with the methods, kits, combinations, and composition of the present invention, include, but are not limited to, rolipram and rolipram derivatives such as RO20-1724, nitraquazone and nitraquazone derivatives such as CP-77059 and RS-25344-00, xanthine derivatives such as denbufylline and IC163197, and other compounds such as EMD54622, LAS-31025 and etazolate; or a salt, an ester, a hydrate, an amide, an enantiomer, an isomer, a tautomer, a polymorph, a prodrug, or a derivative thereof. Combinations of the above mentioned type IV phosphodiesterase inhibitors can be used.

Examples of type V phosphodiesterase inhibitors that can be used in conjunction with the methods, kits, combinations, and composition of the present invention, include, but are not limited to, zaprinast, MY5445, TA-179, dipyridamole, and sildenafil; or a salt, an ester, a hydrate, an amide, an enantiomer, an isomer, a tautomer, a polymorph, a prodrug, or a derivative thereof. Other type V phosphodiesterase inhibitors are disclosed in PCT Publication Nos. WO 94/28902 and WO 96/16644. In the one embodiment, an inhibitor of phosphodiesterase type 5 (“PDE5”), such as VIAGRA® (sildenafil citrate USP) is used. Combinations of the above mentioned type V phosphodiesterase inhibitors can be used.

The phosphodiesterase inhibitors described in PCT Publication No. WO 96/16644 include griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivative, imidazoquinoxalinone derivatives or aza analogues thereof, phenylpyridone derivatives, and others. Specific examples of the phosphodiesterase inhibitors disclosed in WO 96/16644 include 1,3-dimethyl-5-benzylpyrazolo[4,3-d]pyrimidine-7-one, 2-(2-propoxyphenyl)-6-purinone, 6-(2-propoxyphenyl)-1,2-dihydro-2-oxypyridine-3-carboxamide, 2-(2-propoxyphenyl)-pyrido[2,3-d]pyrimid-4(3H)-one, 7-methylthio-4-oxo-2-(2-propoxyphenyl)-3,4-dihydro-pyrimido[4,5-d]pyrimidine, 6-hydroxy-2-(2-propoxyphenyl)pyrimidine-4-carboxamide, 1-ethyl-3-methylimidazo[1,5a]quinoxalin-4(5H)-one, 4-phenylmethylamino-6-chloro-2-(1-imidazoloyl)quinazoline, 5-ethyl-8-[3-(N-cyclohexyl-N-methylcarbamoyl)-propyloxy]-4,5-dihydro-4-oxo-pyrido[3,2-e]-pyrrolo[1,2-a]pyrazine, 5′-methyl-3′-(phenylmethyl)spiro[cyclopentane-1,7′(8′H)-(3′H)-imidazo[2,1b]purin]4′(5′H)-one, 1-[6-chloro-4-(3,4-methylenedioxybenzyl)-aminoquinazolin-2-yl)piperidine-4-carboxylic acid, (6R,9S)-2-(4-trifluoromethyl-phenyl)methyl-5-methyl-3,4,5,6a,7,8,9,9a-octahydr ocyclopent[4,5]-midazo[2,1-b]-purin-4-one, 1t-butyl-3-phenylmethyl-6-(4-pyridyl)pyrazolo[3,4-d]-pyrimid-4-one, 1-cyclopentyl-3-methyl-6-(4-pyridyl)-4,5-dihydro-1H-pyrazolo[3,4-d]pyrimid-4-one, 2-butyl-1-(2-chlorobenzyl)6-ethoxy-carbonylbenzimidaole, and 2-(4-carboxypiperidino)-4-(3,4-methylenedioxy-benzyl)amino-6-nitroquinazoline, and 2-phenyl-8-ethoxycycloheptimidazole; or a salt, an ester, a hydrate, an amide, an enantiomer, an isomer, a tautomer, a polymorph, a prodrug, or a derivative thereof. Combinations of the above mentioned phosphodiesterase inhibitors can be used.

Still other type V phosphodiesterase inhibitors useful in conjunction with the present invention include: IC-351 (tadalafil (Cialis®)); 4-bromo-5-(pyridylmethylamino)-6-[3-(4-chlorophenyl)propoxy]-3(2H)pyridazinone; 1-[4-[(1,3-benzodioxol-5-ylmethyl)amiono]-6-chloro-2-quinazolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]imidazo[2,1-b]purin-4-one; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5-(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); and Sch-51866; or a salt, an ester, a hydrate, an amide, an enantiomer, an isomer, a tautomer, a polymorph, a prodrug, or a derivative thereof. Combinations of the above mentioned type IV phosphodiesterase inhibitors can be used.

The compounds described in PCT Publication No. WO 94/28902 are pyrazolopyrimidinones. Examples of these inhibitor compounds include 5-(2-ethoxy-5-morpholinoacetylphenyl)-1-methyl-3-n-propyl-1,6-dihydro-7H-pyrazolo[4,3-d]pyrimidin-7-one, 5-(5-morpholinoacetyl-2-n-propoxyphenyl)-1-methyl-3-n-propyl-1,6-dihydro-7-H-pyrazolo[4,3-d]pyrimidin-7-one, 5-[2-ethoxy-5-(4-methyl-1-piperazinylsulfonyl)-phenyl1-methyl-3-n-propyl-1,6-dihydro-7H-pyrazolo[4,3-d]pyrimidin-7-one, 5-[2-allyloxy-5-(4-methyl-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-propyl)-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-hydroxyethyl)-1-piperazinylsulfonyl)phenyl]-1-methyl-3-n-propyl-1,6-dihydro-7H-pyrazolo[4,3-d]pyrimidin-7-one, 5-[5-[4-(2-hydroxyethyl)-1-piperazinylsulfonyl]-2-n-propoxyphenyl]-1-methyl-3-n-propyl-1,6-dihydro-7H-pyrazolo[4,3-d]pyrimidin-7-one, 5[2-ethoxy-5-(4-methyl-1-piperazinylcarbonyl)phenyl]-1-methyl-3-n-propyl-1,6-dihydro-7H-pyrazolo[4,3-d]pyrimidin-7-one, and 5-[2-ethoxy-5-(1-methyl-2-imidazolyl)phenyl]-1-methyl-3-n-propyl-1,6-dihydro-7H-pyrazolo[4,3-d]pyrimidin-7-one; or a salt, an ester, a hydrate, an amide, an enantiomer, an isomer, a tautomer, a polymorph, a prodrug, or a derivative thereof. Combinations of the above mentioned pyrazolopyrimidinones can be used.

Other phosphodiesterase inhibitors that may be used in the method of this invention include nonspecific phosphodiesterase inhibitors such as theophylline, IBMX, pentoxifylline and papaverine, and direct vasodilators such as hydralazine.

Combinations of the above mentioned agents for improving sexual performance can be used in the methods, kits, combinations, and composition of the present invention.

The therapeutic compounds which make up the combination therapy may be a combined dosage form or in separate dosage forms intended for substantially simultaneous percutaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by percutaneous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel. Decker, New York, N.Y., 1980.

In one embodiment of the present invention, the method of identifying a subject having, or at risk of developing, a testosterone-deficient disorder is determined by the blood serum levels of the testosterone (or any other hormone) in the subject, which can be accomplished by a simple blood test. For example, a subject's blood is evaluated for the hormone levels and those levels are compared to the optimal or pre-determined physiological levels. In one embodiment the target serum level concentration of free-testosterone is about 0.1 pg to about 10 μg/ml blood serum; the target serum level concentration of progesterone is about 0.25 μg to about 75 μg/ml blood serum; and/or the target serum level concentration of estrogen is about 1 μg to about 1000 μg/ml blood serum. In another embodiment of the present invention, the target serum level concentration of free testosterone is about 3 pg/ml blood serum; the target serum level concentration of progesterone is about 10 μg to about 25 μg/ml blood serum; and/or the target serum level concentration of estrogen is about 100 μg to about 200 μg/ml blood serum. Other risk factors described above can also be used in identifying a subject having, or at risk of developing, a testosterone-deficient disorder.

Based on the comparison of a patient's hormone levels with the targeted or pre-determined physiological levels, a dosage regimen is established for the patient for the replenishment of the level of deficient hormones to targeted physiological levels. It is further contemplated, that after the initial evaluation and the establishment of the regimen, a patient is monitored every 7 to 30 days, by a similar blood test, until the patient attains the targeted or pre-determined physiological level, and the dosages of hormone administration are adjusted accordingly. Once the target levels are established, the regimen directs that the patient continue to follow the established dosage of supplemental hormones indefinitely to maintain the targeted or pre-determined physiological levels. Periodic blood tests are subsequently administered to assure that the targeted or pre-determined physiological levels are maintained.

In one embodiment of the present invention, a pharmaceutical composition comprising testosterone in a hydroalcoholic gel dosage form upon percutaneous administration in a testosterone dosage amount of about 0.4 mg to about 0.5 mg to an adult female premenopausal human subject with a below-normal free testosterone plasma concentration, exhibits a 24 hour testosterone pharmacokinetic profile having at least one of: (i) a Cₘₐₓ not less than about 39 ng total testosterone per dL serum; (ii) a Cₘᵢₙ not less than about 19 ng total testosterone per dL serum; (iii) a C_{avg} not less than about 30 ng total testosterone per dL serum; (iv) a Cₘₐₓ not less than about 2.6 ng free testosterone per dL serum; (v) a Cₘᵢₙ not less than about 1.4 ng free testosterone per dL serum; or (vi) a C_{avg} not less than about 2.1 ng free testosterone per dL serum; with the dosage amount being administered in one to a plurality of dosage unit forms. In another embodiment, this testosterone composition is administered for 7 consecutive days, and on day 7 exhibits a 24 hour testosterone pharmacokinetic profile having at least one of: (i) a Cₘₐₓ not less than about 89 ng total testosterone per dL serum; (ii) a Cₘᵢₙ not less than about 62 ng total testosterone per dL serum; (iii) a C_{avg} not less than about 76 ng total testosterone per dL serum; (iv) a Cₘₐₓ not less than about 6.5 ng free testosterone per dL serum; (v) a Cₘᵢₙ not less than about 5.1 ng free testosterone per dL serum; or (vi) a C_{avg} not less than about 5.6 ng free testosterone per dL serum.

In another embodiment of the present invention, a pharmaceutical composition comprising testosterone in a hydroalcoholic gel dosage form is percutaneously administered to an adult female premenopausal human subject with a below-normal free testosterone plasma concentration in a dose sufficient to result in a 24 hour testosterone pharmacokinetic profile having at least one of: (i) a Cₘₐₓ not less than about 52 ng total testosterone per dL serum; (ii) a Cₘᵢₙ not less than about 18 ng total testosterone per dL serum; (iii) a C_{avg} not less than about 35 ng total testosterone per dL serum; (iv) a Cₘₐₓ not less than about 5.5 ng free testosterone per dL serum; (v) a Cₘᵢₙ not less than about 1.8 ng free testosterone per dL serum; or (vi) a C_{avg} not less than about 3.5 ng free testosterone per dL serum; with the dosage amount being administered in one to a plurality of dosage unit forms. In another embodiment, this testosterone composition is administered for 7 consecutive days, and on day 7 exhibits a 24 hour testosterone pharmacokinetic profile having at least one of: (i) a Cₘₐₓ not less than about 88 ng total testosterone per dL serum; (ii) a Cₘᵢₙ not less than about 43 ng total testosterone per dL serum; (iii) a C_{avg} not less than about 66 ng total testosterone per dL serum; (iv) a Cₘₐₓ not less than about 7.8 ng free testosterone per dL serum; (v) a Cₘᵢₙ not less than about 3.5 ng free testosterone per dL serum; or (vi) a C_{avg} not less than about 5.6 ng free testosterone per dL serum. In one embodiment, the dose of hydroalcoholic gel administers about 0.8 mg to about 0.9 mg testosterone to the subject.

In one embodiment of the present invention, a pharmaceutical composition comprising testosterone in a hydroalcoholic gel dosage form is administered to an adult female postmenopausal human subject with a below-normal free testosterone plasma concentration in a dose sufficient to result in a 24 hour testosterone pharmacokinetic profile having at least one of: (i) a Cₘₐₓ not less than about 19 ng total testosterone per dL serum; (ii) a Cₘᵢₙ not less than about 8 ng total testosterone per dL serum; (iii) a C_{avg} not less than about 14 ng total testosterone per dL serum; (iv) a Cₘₐₓ not less than about 5.5 ng free testosterone per dL serum; (v) a Cₘᵢₙ not less than about 1.8 ng free testosterone per dL serum; or (vi) a C_{avg} not less than about 3.5 ng free testosterone per dL serum; with the dosage amount being administered in one to a plurality of dosage unit forms. In another embodiment, this testosterone composition is administered for 7 consecutive days, and on day 7 exhibits a 24 hour testosterone pharmacokinetic profile having at least one of: (i) a Cₘₐₓ not less than about 44 ng total testosterone per dL serum; (ii) a Cₘᵢₙ not less than about 21 ng total testosterone per dL serum; (iii) a C_{avg} not less than about 32 ng total testosterone per dL serum; (iv) a Cₘₐₓ not less than about 7.8 ng free testosterone per dL serum; (v) a Cₘᵢₙ not less than about 3.5 ng free testosterone per dL serum; or (vi) a C_{avg} not less than about 5.6 ng free testosterone per dL serum. In one embodiment, the dose of hydroalcoholic gel administers about 0.4 mg to about 0.5 mg testosterone to the subject.

In another embodiment of the present invention, a pharmaceutical composition comprising testosterone in a hydroalcoholic gel dosage form is percutaneously administered to an adult female premenopausal human subject with a below-normal free testosterone plasma concentration, exhibits a 24 hour testosterone pharmacokinetic profile having at least one of: (i) a Cₘₐₓ not less than about 61 ng total testosterone per dL serum; (ii) a Cₘᵢₙ not less than about 10 ng total testosterone per dL serum; (iii) a C_{avg} not less than about 34 ng total testosterone per dL serum; (iv) a Cₘₐₓ not less than about 2.6 ng free testosterone per dL serum; (v) a Cₘᵢₙ not less than about 0.8 ng free testosterone per dL serum; or (vi) a C_{avg} not less than about 1.7 ng free testosterone per dL serum; with the dosage amount being administered in one to a plurality of dosage unit forms. In another embodiment, this testosterone composition is administered for 7 consecutive days, and on day 7 exhibits a 24 hour testosterone pharmacokinetic profile having at least one of: (i) a Cₘₐₓ not less than about 106 ng total testosterone per dL serum; (ii) a Cₘᵢₙ not less than about 40 ng total testosterone per dL serum; (iii) a C_{avg} not less than about 64 ng total testosterone per dL serum; (iv) a Cₘₐₓ not less than about 5.6 ng free testosterone per dL serum; (v) a Cₘᵢₙ not less than about 2.3 ng free testosterone per dL serum; or (vi) a C_{avg} not less than about 3.4 ng free testosterone per dL serum. In one embodiment, this composition comprises about 0.1% to about 10% testosterone, or a salt, an ester, a hydrate, an amide, an enantiomer, an isomer, a tautomer, a polymorph, a prodrug, or a derivative thereof; about 30% to about 98% of an alcohol selected from the group consisting of ethanol or isopropanol; about 0.1% to about 5% isopropyl myristate; about 0.1% to about 10% 0.1 N sodium hydroxide; and about 0.1% to about 5% of a gelling agent; wherein the percentages are on a weight to weight basis of the composition and the sum of components of the composition is about 100 weight percent. In one embodiment, the dose of hydroalcoholic gel administers about 0.8 mg to about 0.9 mg testosterone to the subject.

In yet another embodiment of the present invention, the composition comprises about 1% testosterone, or a salt, an ester, a hydrate, an amide, an enantiomer, an isomer, a tautomer, a polymorph, a prodrug, or a derivative thereof; about 0.8% to about 1% polyacrylic acid; about 0.5% isopropyl myristate; about 4% to about 5% 0.1 N sodium hydroxide; and about 72% to about 73% ethanol (95% v/v).

In yet another embodiment, the amount of the composition administered to a subject is a 0.44 g dose delivering about 4.4 mg of testosterone to the skin.

In yet another embodiment, the amount of the composition administered to a subject is a 0.88 g dose delivering about 8.8 mg of testosterone to the skin.

In one embodiment, a method for treating a condition or disorder where treatment with testosterone is indicated comprises percutaneously administering a composition of the present invention to a subject in need of such treatment. Such conditions or disorders include, for example, hypogonadism, sexual dysfunction, decreased libido, hypercholesterolemia, abnormal electrocardiograms, vasomotor symptoms, diabetic retinopathy, hyperglycemia, hyperinsulinemia, hypoinsulinemia, increased percentage of body fat, hypertension, obesity, osteoporosis, osteopenia, vaginal dryness, thinning of the vaginal wall, menopausal symptoms and hot flashes, cognitive dysfunction, cardiovascular disease, central nervous system disorders, Alzheimer's disease, dementia, cataracts, cervical cancer, uterine cancer, breast cancer, depression, improper embryo implantation in the uterus, premature ovarian failure, and premature menopause.

In another embodiment of the present invention, a method of treating, preventing or reducing the risk of developing a testosterone-deficient disorder in an adult subject in need thereof, comprises (i) identifying an adult female premenopausal human subject having a free testosterone plasma concentration of less than about 0.2 ng per dL serum to about 0.5 ng per dL serum; (ii) administering an amount of a composition of claim 1 to an area of skin of the subject, which delivers a therapeutically-effective amount of testosterone to the blood serum of the subject such that the testosterone-deficient disorder or the risk of developing a testosterone-deficient disorder is reduced.

The present invention is further illustrated by the following examples, which should not be construed as limiting in any way. In the below example, it is assumed that normal cycling women produce approximately 300 μg of testosterone per day, and their serum testosterone levels generally range from about 20 ng/dL to about 80 ng/dL averaging about 40 ng/dL. Bilateral oophorectomy in premenopausal women reduces testosterone production by approximately 50%, resulting in an average total serum level of approximately 20 ng/dL. From a physiological perspective, testosterone therapy in surgically menopausal women who, for example, experience female sexual dysfunction, is to replace the missing ovarian testosterone production of approximately 150 μg per day and restore the levels of testosterone and its active androgenic metabolite dihydrotestosterone (DHT) to their previous levels within the normal physiological range.

### EXAMPLES

The following examples are provided for exemplification of the present invention and are not intended to be limiting in any way. Unless otherwise indicated, in these examples, testosterone is formulated as a gel for transdermal administration as described above in Table 4.

### Example 1

### Increase in Testosterone Concentrations in Premenopausal Women with Below-Normal Testosterone Concentrations after Administration of 4.4 mg of Testosterone in a 1% Gel Formulation

This example demonstrates the increase in serum testosterone levels after the application of 4.4 mg of transdermal testosterone as a 1% hydroalcoholic gel in premenopausal women with below-normal free testosterone concentrations.

In this example, five premenopausal women between the ages of 18 and 65 years old were enrolled in the study. The subjects were identified as having below-normal free (unbound) testosterone concentrations for their age, with free (unbound) testosterone concentrations at screening ranging from 0.2-0.5 ng/dL. In addition, the subjects could not have received testosterone therapy within 30 days of screening, be pregnant or lactating, be diabetic, or be receiving any anti-depressant therapy.

The women received 0.44 g of a 1% testosterone gel formulation once daily for seven days at approximately 8:00 am. The gel was dispensed from glass bottles using metered pumps. The amount of testosterone in the applied daily doses was 4.4 mg. The gel was applied evenly to the skin of the outer, upper thigh and hip at the same time on each of the seven study days.

Sequential blood samples were obtained on Day 0 (baseline), Day 1 and Day 7 of the treatment for purposes of determining the testosterone pharmacokinetics. Serum collected from the patients was assayed for total (bound+unbound) and unbound testosterone (not bound to either albumin or sex hormone binding globulin (SHBG)) using a validated sensitive and specific radioimmunoassay.

The peak concentration during the dosing interval (Cₘₐₓ) and the lowest concentration (Cₘᵢₙ) during the dosing interval were determined by inspection. The average concentration (C_{avg}) over the dosing interval was calculated as AUC_{(0-24)/24}, where the AUC₍₀₋₂₄₎ was the area under the concentration-time curve over the 24-hour dosing interval as determined using the trapezoidal rule. If an assay result was missing, the AUC was calculated by extending the trapezoid to the next sample time. If the missing assay result was at the beginning or end of the dosing interval on Day 0 or Day 7, the missing value was estimated as equal to the assay result 24 hours earlier or later, based on the assumption that the patient was at steady-state.

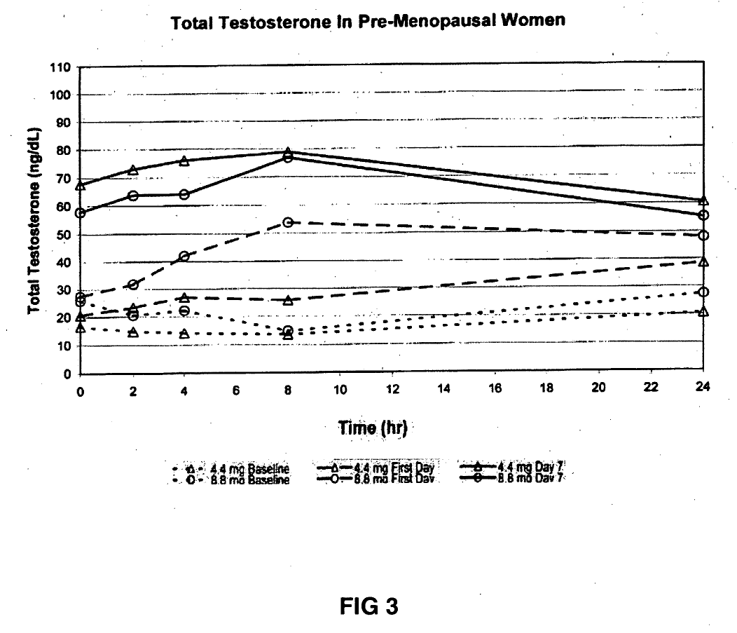

Topically applied testosterone-containing hydroalcoholic gel raised the serum testosterone levels of premenopausal women with below-normal testosterone levels soon after application. By the end of the first day of treatment, serum concentrations of total testosterone and free testosterone had approximately doubled. Treatment once daily for a total of seven days resulted in serum concentrations between 3 and 6 times the pretreatment levels. The total testosterone levels (ng/dL) are shown in FIG. 3.

The increase in maximum (Cₘₐₓ), minimum (Cₘᵢₙ) and average (C_{avg}) total testosterone concentration on Day 1 and Day 7 compared to the maximum (Cₘₐₓ), minimum (Cₘᵢₙ) and average (C_{avg}) total testosterone concentration at baseline (prior to initiating treatment) is summarized in Table 9.

TABLE 9
Mean Total Testosterone Concentration in Premenopausal Women
With Administration of 4.4 mg of Testosterone in a 1% Hydroalcoholic
Gel

| | | Total Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Premenopausal | Day 0 | 21 ± 3 | 13 ± 3 | 16 ± 3 | — |
| Dose 4.4 mg | Day 1 | 39 ± 5 | 19 ± 4 | 30 ± 5 | 14 ± 3 |
| (N = 5) | Day 7 | 89 ± 23 | 62 ± 18 | 76 ± 20 | 59 ± 19 |

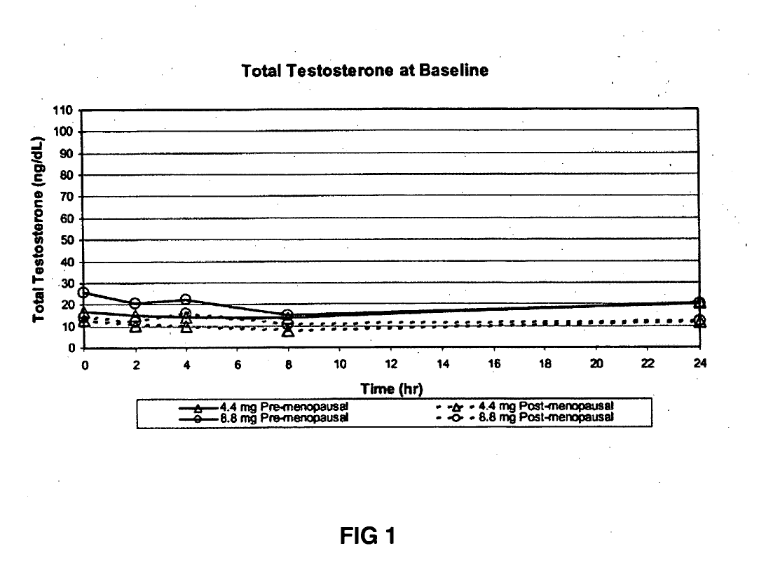

Baseline values for total testosterone average concentration over 24 hours, C_{avg}, are shown in FIG. 1. Baseline values ranged from 9 to 26 ng/mL, with a mean of 16 ng/mL.

On the first day of treatment with 4.4 mg of testosterone gel, the women increased their total testosterone C_{avg} by 37-155% compared to baseline, with a mean increase of 94%.

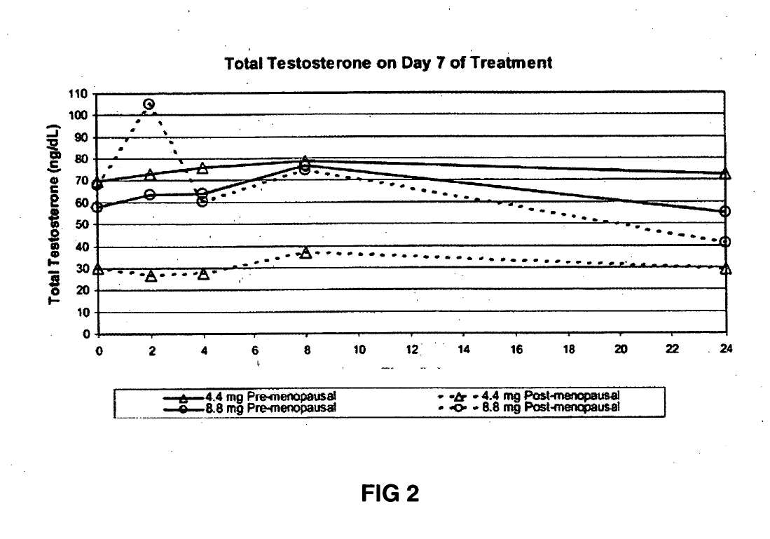

Day 7 values for total testosterone average concentration over 24 hours, C_{avg}, are illustrated in FIG. 2. Application of 4.4 mg of testosterone gel resulted in a mean change from 16 ng/dL to 76 ng/dL in the premenopausal women.

Baseline concentration of total testosterone showed a slight diurnal variation with peak concentrations associated with the early morning sample in the five subjects. Application of testosterone altered this diurnal pattern. Application of the testosterone gel in the early morning at the approximate time of normal maximum testosterone concentrations) resulted in the highest serum concentrations being observed at some other time than early morning in four of the five subjects. Thus, it follows that if patients applied the testosterone gel at night, prior to going to bed, both the timing and the extent of diurnal variation in the patient would probably follow the normal pattern.

In an effort to see if the steady-state testosterone concentrations were predicted by a marker concentration on Day 1, correlations were looked for between concentrations on each of the sample times on Day 1 and either the C_{avg} on Day 7, or the change in C_{avg} between Day 0 and Day 7. The highest correlation was identified between the 24 hr sample on Day 1 and the Day 7 C_{avg} (R²=0.35). The relationship indicates that the steady-state C_{avg} on Day 7 can be predicted to be approximately 23 ng/dL greater than the testosterone serum concentration measured 24 hours after application of the first dose, i.e., just prior to the second application of the gel.

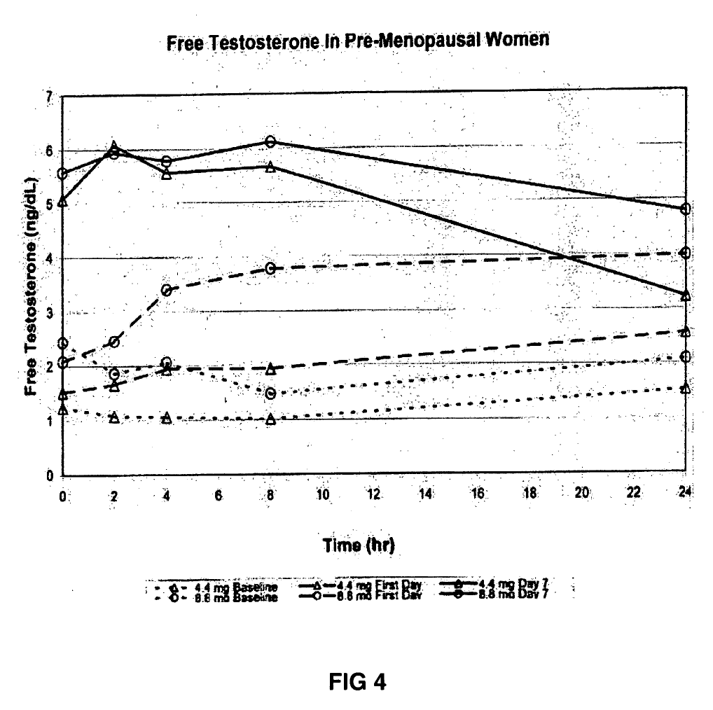

Serum concentrations of free testosterone increased in parallel with total testosterone concentration, as shown in FIG. 4. The increase in maximum (Cₘₐₓ), minimum (Cₘᵢₙ) and average (C_{avg}) free testosterone concentration on Day 1 and Day 7 compared to the maximum (Cₘₐₓ), minimum (Cₘᵢₙ) and average (C_{avg}) free testosterone concentration at baseline (prior to initiating treatment) is summarized in Table 10.

TABLE 10
Mean Free Testosterone Concentration in Premenopausal Women with
Administration of 4.4 mg of Testosterone in a 1% Hydroalcoholic Gel

| | | Free Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Premenopausal | Day 0 | 1.6 ± 0.6 | 1.0 ± 0.4 | 1.2 ± 0.5 | — |
| Dose 4.4 mg | Day 1 | 2.6 ± 0.8 | 1.4 ± 0.6 | 2.1 ± 0.7 | 0.9 ± 0.3 |
| (N = 5) | Day 7 | 6.5 ± 2.6 | 5.1 ± 2.5 | 5.6 ± 2.4 | 4.4 ± 1.9 |

The mean change in free testosterone compared to baseline on the first day of treatment was 92%. On Day 7, the mean C_{avg} for free testosterone ranged from over 2 times to about 5 times the pretreatment level, with one patient showing a 10-fold increase in free testosterone on Day 7. Application of 4.4 mg of testosterone gel increased the mean C_{avg} for free testosterone to 5.6 ng/dL in the premenopausal subjects.

Maximum (Cₘₐₓ), minimum (Cₘᵢₙ) and average (C_{avg}) testosterone free fraction on Day 1 and Day 7 compared to the maximum (Cₘₐₓ), minimum (Cₘᵢₙ) and average (C_{avg}) testosterone free fraction at baseline (prior to initiating treatment) is summarized in Table 11.

TABLE 11
Mean Testosterone Free Fraction in Premenopausal Women with
Administration of 4.4 mg of Testosterone in a 1% Hydroalcoholic Gel

| | | Free Fraction | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Premenopausal | Day 0 | 7.3% ± 1.3% | 5.6% ± 1.6% | 6.3% ± | — |
| Dose 4.4 mg | | | | 1.5% | |
| (N = 5) | Day 1 | 6.7% ± 1.6% | 6.0% ± 1.5% | 6.3% ± | 0.1% ± |
| | | | | 1.5% | 0.2% |
| | Day 7 | 7.7% ± 1.7% | 7.0% ± 1.5% | 7.4% ± | 1.1% ± |
| | | | | 1.6% | 0.6% |

As shown in Table 11, in premenopausal women applying 4.4 mg testosterone, the mean average free fraction changed from 6.3% to 7.4%, with the average change from Day 0 to Day 7 being an increase of 1.1%. The C_{avg} for the free fraction at baseline ranged from 2.1% to 11% in the five subjects. C_{avg} for the free fraction on Day 7 showed a similar degree of variation with values ranging from 1.8% to 11.3%.

### Example 2

### Increase in Testosterone Concentrations in Premenopausal Women with Below-Normal Testosterone Concentrations after Administration of 8.8 mg of Testosterone in a 1% Hydroalcoholic Gel Formulation

This example demonstrates the increase in serum testosterone levels after the application of 8.8 mg of transdermal testosterone as a 1% hydroalcoholic gel in premenopausal women with below-normal free testosterone concentrations.

In this example, five premenopausal women between the ages of 18 and 65 years old were enrolled in the study as above. The women received 0.88 g of a 1% testosterone gel formulation (containing 8.8 mg of testosterone) once daily for seven days. With the exception of the amount of testosterone administered to the subjects, all aspects of the study were identical to those described in Example 1.

Topical application of 8.8 mg of testosterone-containing hydroalcoholic gel raised the serum testosterone levels of premenopausal women with below-normal testosterone levels. By the end of the first day of treatment, serum concentrations of total testosterone and free testosterone had approximately doubled. Treatment once daily for a total of seven days resulted in serum concentrations between 2 and 7 times the pretreatment levels. The total testosterone levels (ng/dL) are shown in FIG. 3.

The increase in maximum (Cₘₐₓ), minimum (Cₘᵢₙ) and average (C_{avg}) total testosterone concentration on Day 1 and Day 7 compared to the maximum (Cₘₐₓ), minimum (Cₘᵢₙ) and average (C_{avg}) total testosterone concentration at baseline (prior to initiating treatment) is summarized in Table 12.

TABLE 12
Mean Total Testosterone Concentration in Premenopausal Women
With Administration of 8.8 mg of Testosterone in a 1% Hydroalcoholic
Gel

| | | Total Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Premenopausal | Day 0 | 30 ± 8 | 13 ± 4 | 18 ± 4 | — |
| Dose 8.8 mg | Day 1 | 52 ± 6 | 18 ± 5 | 35 ± 7 | 16 ± 4 |
| (N = 5) | Day 7 | 88 ± 13 | 43 ± 9 | 66 ± 12 | 47 ± 13 |

Baseline values for total testosterone average concentration over 24 hours, C_{avg}, are shown in FIG. 1. Baseline values ranged from 5 to 30 ng/mL, with a mean of 18 ng/mL.

On the first day of treatment with 8.8 mg of testosterone gel, the testosterone C_{avg} was increased by 38-220% compared to baseline, with a mean increase of 109%.

Day 7 values for total testosterone average concentration over 24 hours, C_{avg}, are illustrated in FIG. 2. Application of 8.8 mg of testosterone gel resulted in a mean change from 18 ng/dL to 66 ng/dL in the subjects.

Baseline concentration of total testosterone showed a slight diurnal variation with peak concentrations associated with the early morning sample in one of the five subjects. Application of testosterone altered the diurnal pattern of the subjects.

As in Example 1, serum concentrations of free testosterone increased in parallel with total testosterone concentration. (FIG. 4). The increase in maximum (Cₘₐₓ), minimum (Cₘᵢₙ) and average (C_{avg}) free testosterone concentration on Day 1 and Day 7 compared to the maximum (Cₘₐₓ), minimum (Cₘᵢₙ) and average (C_{avg}) free testosterone concentration at baseline is summarized in Table 13.

TABLE 13
Mean Free Testosterone Concentration in Premenopausal Women With
Administration of 8.8 mg of Testosterone in a 1% Hydroalcoholic Gel

| | | Free Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Premenopausal | Day 0 | 2.7 ± 0.9 | 1.3 ± 0.6 | 1.7 ± 0.6 | — |
| Dose 8.8 mg | Day 1 | 5.5 ± 1.0 | 1.8 ± 0.6 | 3.5 ± 0.8 | 1.5 ± 0.2 |
| (N = 5) | Day 7 | 7.8 ± 1.1 | 3.5 ± 0.6 | 5.6 ± 0.7 | 3.9 ± 1.0 |

The mean free testosterone C_{avg} increase on the first day of treatment, compared with baseline, was 110%. On Day 7, the C_{avg} for free testosterone ranged from nearly 2 times to almost 8 times the pretreatment level. Application of 8.8 mg of testosterone gel increased the C_{avg} of the free testosterone from 1.7 ng/dL to 5.6 ng/dL in the subjects on Day 7.

Maximum (Cₘₐₓ), minimum (Cₘᵢₙ) and average (C_{avg}) testosterone free fraction on Day 1 and Day 7 compared to the maximum (Cₘₐₓ), minimum (Cₘᵢₙ) and average (C_{avg}) testosterone free fraction at baseline with administration of 8.8 mg of testosterone to premenopausal women is summarized in Table 14.

TABLE 14
Mean Testosterone Free Fraction in Premenopausal Women With
Administration of 8.8 mg of Testosterone in a 1% Hydroalcoholic Gel

| | | Free Fraction | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Premeno- | Day 0 | 10.5% ± | 9.4% ± | 9.9% ± | — |
| pausal | | 2.5% | 2.4% | 2.4% | |
| Dose | Day 1 | 11.9% ± | 10.6% ± | 11.1% ± | −0.1% ± 0.1% |
| 8.8 mg | | 2.7% | 2.6% | 2.7% | |
| (N = 5) | Day 7 | 10.1% ± | 9.2% ± | 9.6% ± | −0.3% ± 1.0% |
| | | 2.0% | 2.1% | 2.0% | |

As shown in Table 14, in premenopausal women applying 8.8 mg testosterone, the mean average free fraction changed from 9.9% to 9.6%, with the average change from Day 0 to Day 7 being an increase of 1.1%. Free fraction baseline ranged from 4.7% to 17.5% in the five subjects. Free fractions on Day 7 showed a similar degree of variation with values ranging from 1.8% to 11.3%.

### Example 3

### Increase in Free Testosterone Concentrations in Postmenopausal Women with Below-Normal Free Testosterone Concentrations after Administration of 4.4 mg of Testosterone in a 1% Hydroalcoholic Gel Formulation

This example demonstrates the increase in serum testosterone levels after the application of 4.4 mg of transdermal testosterone as a 1% hydroalcoholic gel in postmenopausal women with below-normal free testosterone concentrations.

In this example, four postmenopausal women between the ages of 18 and 65 years old were enrolled in the study. The subjects were identified as having below-normal free (unbound) testosterone concentrations for their age, with free (unbound) testosterone concentrations at screening ranging from 0.2-0.5 ng/dL. The women received 0.44 g of a 1% testosterone gel formulation (containing 4.4 mg of testosterone) once daily for seven days. All aspects of the study were identical to those described in Example 1.

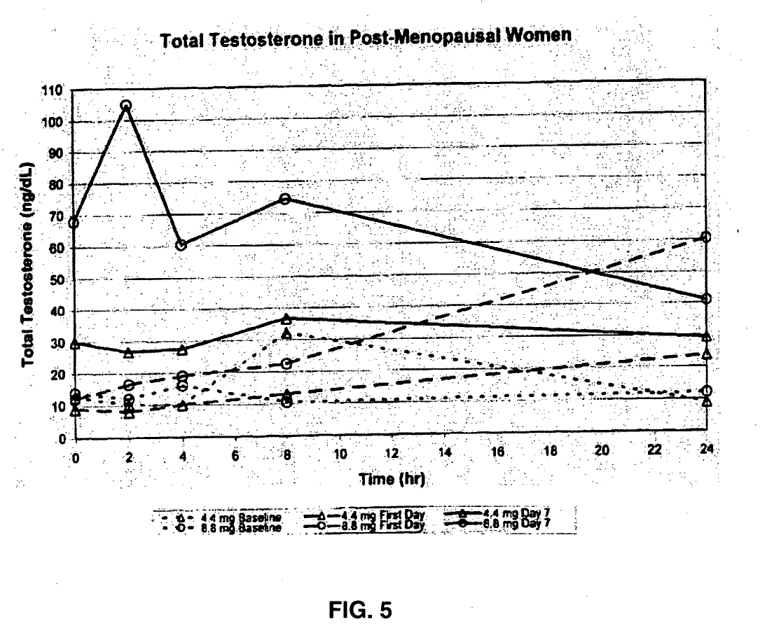

Topical application of 4.4 mg of testosterone-containing hydroalcoholic gel raised the serum testosterone levels of postmenopausal women with below-normal testosterone levels soon after application, as shown in FIG. 5. By the end of the first day of treatment, serum concentrations of total testosterone and free testosterone were approximately 1.5 to 2 times greater. Treatment once daily for a total of seven days resulted in serum concentrations between 2 and 5 times the pretreatment levels.

The increase in maximum (Cₘₐₓ), minimum (Cₘᵢₙ) and average (C_{avg}) total testosterone concentration on Day 1 and Day 7 compared to the maximum (Cₘₐₓ), minimum (Cₘᵢₙ) and average (C_{avg}) total testosterone concentration at baseline is summarized in Table 15.

TABLE 15
Mean Total Testosterone Concentration in Postmenopausal Women
With Administration of 4.4 mg of Testosterone in a 1% Hydroalcoholic
Gel

| | | Total Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Postmenopausal | Day 0 | 13 ± 3 | 10 ± 3 | 11 ± 3 | — |
| Dose 4.4 mg | Day 1 | 19 ± 5 | 8 ± 2 | 14 ± 4 | 5 ± 2 |
| (N = 4) | Day 7 | 44 ± 13 | 21 ± 4 | 32 ± 9 | 21 ± 7 |

Baseline values for total testosterone average concentration over 24 hours, C_{avg}, are shown in FIG. 1. Baseline values for total testosterone C_{avg} ranged from 6 to 18 ng/mL, with a mean of 11 ng/mL.

On the first day of treatment with 4.4 mg of testosterone gel, the testosterone C_{avg} was increased by 50-83% compared to baseline, with a mean increase of 61%.

Day 7 values for total testosterone average concentration over 24 hours, C_{avg}, are illustrated in FIG. 2. Day 7 levels were 2 to 4.5 times the pretreatment levels, with a mean change in total testosterone average concentration from 11 ng/dL to 32 ng/dL in the subjects.

Baseline concentration of total testosterone showed a slight diurnal variation with peak concentrations associated with the early morning sample in all of the subjects, and application of the testosterone gel in the early morning (at the approximate time of normal maximum testosterone concentrations) resulted in the highest serum concentrations being observed at some other time than early morning in each of the subjects as well.

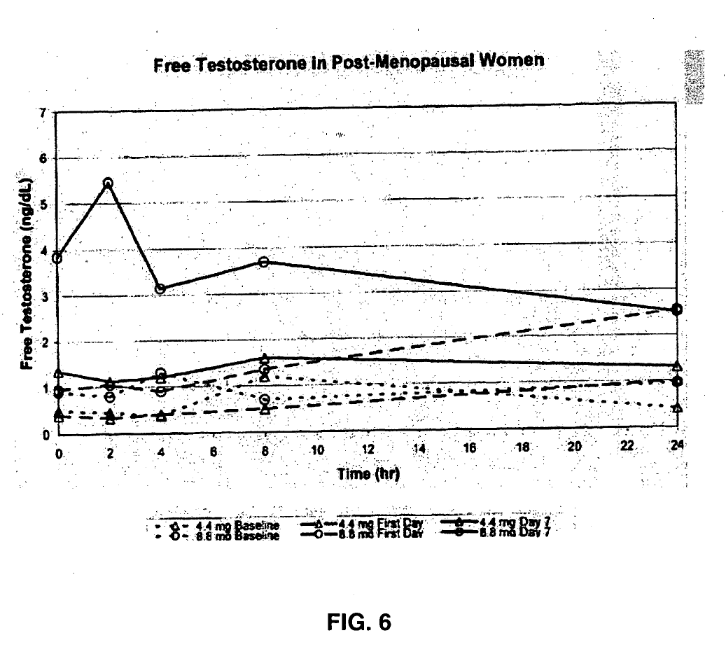

Serum concentrations of free testosterone increased along with the total testosterone concentration after administration of 4.4 mg of testosterone gel to the postmenopausal women, as shown in FIG. 6. The increase in maximum (Cₘₐₓ), minimum (Cₘᵢₙ) and average (C_{avg}) free testosterone concentration on Day 1 and Day 7 compared to the maximum (Cₘₐₓ), minimum (Cₘᵢₙ) and average (C_{avg}) free testosterone concentration at baseline (prior to initiating treatment) is summarized in Table 16.

TABLE 16
Mean Free Testosterone Concentration in Postmenopausal Women with
Administration of 4.4 mg of Testosterone in a 1% Hydroalcoholic Gel

| | | Free Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Postmenopaus- | Day 0 | 0.5 ± 0.9 | 0.4 ± 0.1 | 0.4 ± 0.1 | — |
| al Dose 4.4 mg | Day 1 | 5.5 ± 1.0 | 1.8 ± 0.6 | 3.5 ± 0.8 | 1.5 ± 0.2 |
| (N = 4) | Day 7 | 7.8 ± 1.1 | 3.5 ± 0.6 | 5.6 ± 0.7 | 3.9 ± 1.0 |

The free testosterone mean C_{avg} increased from 0.4 ng/dL at baseline to 3.5 ng/dL on the first day of treatment and to 5.6 ng/dL on the seventh day of treatment. On Day 7, the mean C_{avg} for free testosterone ranged from over 2 times to over 5 times the pretreatment level.

Maximum (Cₘₐₓ), minimum (Cₘᵢₙ) and average (C_{avg}) testosterone free fraction on Day 1 and Day 7 compared to the maximum (Cₘₐₓ), minimum (Cₘᵢₙ) and average (C_{avg}) testosterone free fraction at baseline (prior to initiating treatment) is summarized in Table 17.

TABLE 17
Mean Testosterone Free Fraction in Postmenopausal Women with
Administration of 4.4 mg of Testosterone in a 1% Hydroalcoholic Gel

| | | Free Fraction | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Postmenopaus- | Day 0 | 5.3% ± 1.1% | 3.6% ± 0.5% | 4.1% ± | — |
| al Dose 4.4 mg | | | | 0.5% | |
| (N = 4) | Day 1 | 4.9% ± 0.9% | 3.8% ± 0.6% | 4.4% ± | 0.1% ± |
| | | | | 0.7% | 0.2% |
| | Day 7 | 4.9% ± 0.5% | 4.2% ± 0.3% | 4.7% ± | 0.6% ± |
| | | | | 0.5% | 0.1% |

As illustrated in Table 17, in postmenopausal women applying 4.4 mg testosterone, the mean average free fraction changed from 4.1% to 4.7%.

### Example 4

### Increase in Testosterone Concentrations in Postmenopausal Women with Below-Normal Testosterone Concentrations after Administration of 8.8 mg of Testosterone in a 1% Hydroalcoholic Gel Formulation

This example demonstrates the increase in serum testosterone levels after the application of 8.8 mg of transdermal testosterone as a 1% hydroalcoholic gel in postmenopausal women with below-normal free testosterone concentrations.

In this example, two postmenopausal women between the ages of 18 and 65 years old were enrolled in the study as above. The women received 0.88 g of a 1% testosterone gel formulation (containing 8.8 mg of testosterone) once daily for seven days. The methodology of the study was identical to that described in Example 1.

Topical application of 8.8 mg of testosterone-containing hydroalcoholic gel raised the serum testosterone levels of postmenopausal women with below-normal testosterone levels immediately after application, as shown in FIG. 5. Concentrations of total testosterone and free testosterone increased significantly after the seventh day of administration compared to baseline concentrations. Serum concentrations after seven days of treatment with the testosterone gel increased over twice the baseline concentrations in one subject and over 12 times the baseline concentrations in a second subject.

The values for the maximum (Cₘₐₓ), minimum (Cₘᵢₙ) and average (C_{avg}) total testosterone concentration on Day 1 and Day 7 and the values for the maximum (Cₘₐₓ), minimum (Cₘᵢₙ) and average (C_{avg}) total testosterone concentration at baseline are summarized in Table 18.

TABLE 18
Mean Total Testosterone Concentration in Premenopausal Women
With Administration of 8.8 mg of Testosterone in a 1% Hydroalcoholic
Gel

| | | Total Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Postmenopausal | Day 0 | 16 ± 5 | 8 ± 4 | 11 ± 4 | — |
| Dose 8.8 mg | Day 1 | 61 ± 37 | 10 ± 6 | 34 ± 15 | 22 ± 19 |
| (N = 2) | Day 7 | 106 ± 64 | 40 ± 4 | 64 ± 26 | 52 ± 30 |

Baseline values for total testosterone average concentration over 24 hours, C_{avg}, are shown in FIG. 1. Baseline C_{avg} total testosterone values were 7 ng/dL for the first subject and 16 ng/dL for the second subject. On the first day of treatment with 8.8 mg of testosterone gel, the C_{avg} values increased to 49 ng/dL for the first subject and to 19 ng/dL for the second subject. On the seventh day of treatment, total testosterone C_{avg} values were 89 ng/dL and 38 ng/dL for the first and second subjects, respectively. Day 7 values for the total testosterone C_{avg} are illustrated in FIG. 2.

Baseline concentration of total testosterone showed a slight diurnal variation with peak concentrations associated with the early morning sample in one of the subjects. Application of testosterone altered the diurnal pattern of the subjects' testosterone levels.

Serum concentrations of free testosterone increased in parallel with total testosterone concentration, as shown in FIG. 6. Maximum (Cₘₐₓ), minimum (Cₘᵢₙ) and average (C_{avg}) free testosterone concentration at baseline, and on Day 1 and Day 7, are summarized in Table 19.

TABLE 19
Mean Free Testosterone Concentration in Postmenopausal Women
With Administration of 8.8 mg of Testosterone in a 1% Hydroalcoholic
Gel

| | | Free Testosterone | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Postmenopaus- | Day 0 | 1.0 ± 0.5 | 0.7 ± 0.4 | 0.8 ± 0.4 | — |
| al Dose 8.8 mg | Day 1 | 2.6 ± 0.9 | 0.8 ± 0.4 | 1.7 ± 0.3 | 0.9 ± 0.7 |
| (N = 2) | Day 7 | 5.6 ± 2.2 | 2.3 ± 0.3 | 3.4 ± 0.6 | 2.6 ± 1.0 |

At baseline, free testosterone mean C_{avg} was 0.4 ng/dL in the first subject and 1.2 in the second subject. On Day 1, the mean C_{avg} increased to 2.0 ng/dL in the first subject and 1.4 in the second subject, and on Day 7, the mean C_{avg} for free testosterone was 4.0 ng/dL in the first subject and 2.8 ng/dL in the second subject.

Maximum (Cₘₐₓ), minimum (Cₘᵢₙ) and average (C_{avg}) testosterone free fractions on baseline, Day 1, and Day 7 are summarized in Table 20.

TABLE 20
Mean Testosterone Free Fraction in Postmenopausal Women With
Administration of 8.8 mg of Testosterone in a 1% Hydroalcoholic Gel

| | | Free Fraction | | | |
|---|---|---|---|---|---|
| | | Cₘₐₓ | Cₘᵢₙ | C_{avg} | Δ C_{avg} |
| | | (ng/dL) | (ng/dL) | (ng/dL) | (ng/dL) |
| Postmenopausal | Day 0 | 9.2% ± | 5.9% ± | 7.1% ± | — |
| Dose 8.8 mg | | 0.8% | 1.6% | 0.8% | |
| (N = 2) | Day 1 | 8.8% ± | 5.3% ± | 6.1% ± | −1.0% ± 0.5% |
| | | 1.2% | 1.8% | 1.3% | |
| | Day 7 | 6.4% ± | 5.6% ± | 5.9% ± | −1.2% ± 0.6% |
| | | 1.7% | 1.3% | 1.5% | |

As shown in Table 20, in postmenopausal women applying 8.8 mg testosterone, the mean average free fraction changed from 6.3% at baseline to 4.7% on Day 1 of treatment, and to 4.4% on Day 7 of treatment in the first subject. The mean average free fraction changed from 7.9% at baseline to 7.4% on both Day 1 and Day 7 of treatment in the second subject.

### Example 5

### Menopausal Status and Effect of Exogenous Testosterone Administration on Serum Testosterone Levels

This example demonstrates the effect of exogenous testosterone administration on serum testosterone levels in pre- and post-menopausal women after the application of either 4.4 mg or 8.8 mg of transdermal testosterone as a 1% hydroalcoholic gel. The study was performed as described in Examples 1-4.

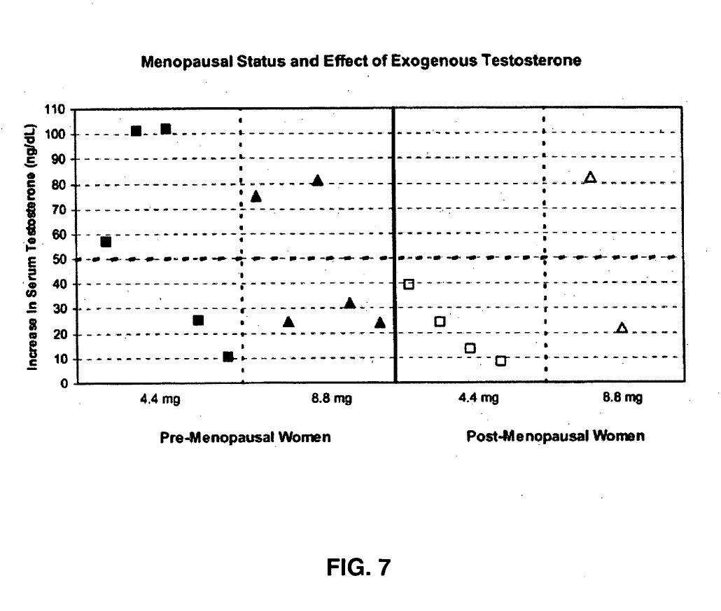

The results of this study are detailed in FIG. 7. FIG. 7 shows that the extent of which the exogenous testosterone increased serum testosterone levels was greater in the pre-menopausal than in the post-menopausal women. The greatest increases in serum testosterone levels were observed in pre-menopausal women receiving the lower dose of testosterone. Five of the six post-menopausal women had serum testosterone increases of less than 50 ng/dL, while only 50% of the premenopausal women fit into that category. The pre-menopausal women did not show a dose-dependence in terms of the extent of the observed increase. The number of post-menopausal women who received the higher dose was too small to identify the presence or absence of a dose-dependence in the post-menopausal women.

### Example 6

### Testosterone Concentrations in Postmenopausal Women after Administration of Testosterone in a 1% Hydroalcoholic Gel Formulation with or without Hormone Replacement Therapy

This example demonstrates the increase in serum testosterone levels in postmenopausal women after the application of either 4.4 mg or 8.8 mg or 13.2 mg of transdermal testosterone as a 1% hydroalcoholic gel with or without hormone replacement therapy.

In this example, 24 postmenopausal women who were 51 years of age or older were enrolled in the study. Menopause was defined as the cessation of menstruation for more than one year and/or a FSH level greater than 30 U/L in women who were not on estrogen replacement therapy. The participants had to have a serum testosterone level (early morning) of less than 33 ng/dL and a normal PAP smear and mammogram in the preceding 12 months. Subjects were divided into two groups: Group I included 13 healthy, postmenopausal women who had not been on hormone replacement therapy for at least three months, and Group II included 13 postmenopausal women who had been treated for at least the preceding three months with a stable hormone replacement regimen. Women who had undergone oophorectomy were not eligible for the study.

The study consisted of a screening period, a 24-hour baseline-sampling period, and a 5-week treatment period, where subjects applied either 4.4 mg, 8.8 mg, or 13.2 mg daily of a 1% testosterone hydroalcoholic gel (Relibra®, Unimed Pharmaceuticals, Inc., Marietta, Ga.).

Serum total testosterone levels were measured using a radioimmunoassay that uses iodinated testosterone as a tracer. Javanbakht, M., et al., J. Clin. Endocrinol. Metab., Vol. 85, No. 7, 2000, pp. 2395-401. This assay has a sensitivity of 0.44 ng/dL and intra- and inter-assay coefficients of variation of 13.2 and 8.2% respectively. Free testosterone levels were measured by a sensitive equilibrium dialysis method (Sinha-Hikim, I, et al., J. Clin. Endocrinol. Metab., Vol. 83, 1998, pp. 1312-1318) optimized to measure low concentrations with precision and accuracy. Two hundred μL serum in the inner compartment was dialyzed against 2.4 mL dialysis buffer that approximates the composition of a protein-free ultrafiltrate of human serum. Id., Dialysis was performed overnight for 16 h at 37° C. Testosterone concentration in the dialysate was measured by a RIA using ¹²⁵I-labeled testosterone. Id. The sensitivity of the free testosterone assay is 0.6 pg/ml (2.0 pmol/L) and intra- and inter-assay coefficients of variation 4.2 and 12.3%, respectively. Serum LH, FSH, and SHBG levels were measured by two site-directed immunofluorometric assays (Delfia-Wallac, Gaithersburg, Md.), with sensitivities of 0.05 U/L, 0.15 U/L, and 6.25 nmol/L, respectively, as described previously. Javanbakht, et al., and Sinha-Hikim, et al., supra. The intra- and inter-assay coefficients of variation were 10.7% and 13.0% for LH, 3.2% and 11.3% for FSH, and 10% and 10.2% for SHBG, respectively. Serum estradiol levels were measured by a radioimmunoassay with a sensitivity of 2.5 pg/mL, and intra- and inter-assay coefficients of variation of 8% and 10%, respectively.

Baseline and pharmacokinetic parameters were averaged across subjects within each group to obtain means, SDs, and SEMs. The bioavailability of testosterone was described as area under the testosterone curve (AUC). The time-average free and total testosterone concentrations during the 24-hour sampling period were computed form the areas under the respective curves, divided by 24 hours. The following pharmacokinetic parameters from the 24-hour profiles of free and total testosterone measured on days 8, 22, and 35 were evaluated: time-average, steady-state concentration (C_{average}), maximum concentration (Cₘₐₓ), minimum concentration (Cₘᵢₙ), time of maximum concentration (Tₘₐₓ), time of minimum concentration (Tₘᵢₙ), and area under the curve (AUC). The C_{average} was computed from 24 AUC by dividing it by 24 hours.

There were two treatment groups: postmenopausal women who received testosterone gel alone and were not on estrogen therapy, and postmenopausal women who were concomitantly receiving a stable hormone replacement regimen that included premarin and medroxyprogesterone. Different doses of testosterone gel were administered in random order. Therefore, we used a three way mixed model analysis of variance (ANOVA) with testosterone dose (4.4, 8.8 or 13.2 mg daily), estrogen (yes or no), and time in hours after gel application as the three factors. A patient effect (nested within treatment) was included as a covariate in the model. In order to simplify the estimation process, the three-way interaction between estrogen treatment, dose, and time was assumed to be zero, but all two-way interactions were used in the analysis, including the appropriate interactions with the nested patient effect. If an F-test revealed a significant effect, then differences between individual groups were analyzed by using the Tukey-Kramer multiple comparison procedure. In order to meet the distributional assumptions of the ANOVA model, serum hormone concentrations were log-transformed before analyses. The analysis of the pharmacokinetic parameters across the dose arms used a two-way, repeated measures analysis of variance, where estrogen treatment was the treatment variable and dose was the repeated factor. The Tukey-Kramer multiple comparison procedure was used for analyzing any significant dose effects.

Baseline characteristics of the subjects were measured. The two groups of women, those who had been on a stable regimen of estrogen replacement and those who were not on estrogen replacement therapy, were not significantly different in terms of age, body weight, height, body mass index and baseline serum total and free testosterone levels. Serum estradiol and SHBG levels were higher, and LH and FSH levels lower in women who were receiving estrogen replacement therapy than in those who were not, as shown in Table 21.

TABLE 21
Baseline Characteristics of Study Participants

| | | Group II |
|---|---|---|
| | Group I | Healthy Postmenopausal |
| | Healthy Postmenopausal | Women Receiving |
| | Women Not Receiving | Hormone Replacement |
| | Hormone Replacement | Therapy for at Least |
| | Therapy | 3 Months |
| Number of Women | 13 | 13 |
| Age (years) | 57 ± 2 | 58 ± 2 |
| Height (cm) | 156 ± 2 | 159 ± 2 |
| Body Weight (kg) | 69 ± 3 | 70 ± 2 |
| BMI (kg/m²) | 28 ± 1 | 27 ± 1 |
| Ethnicity | | |
| White | 0 | 5 |
| Hispanic | 6 | 2 |
| African American | 6 | 4 |
| Asian | 1 | 2 |
| Duration of HRT | N/A | 8 ± 2 |
| (years) | | |
| Type of HRT | | Conjugated equine estro- |
| | | gen (0.625 mg) plus |
| | | medroxyprogesterone |
| | | acetate (2.5 mg) = 11 |
| | | Conjugated equine estro- |
| | | gen (0.625 mg) |
| | | alone* = 1 |
| | | Estrogen patch |
| | | alone* = 1 |
| Time since meno- | 12 ± 2 | 12 ± 3 |
| pause (years) | | |

*These two women had undergone hysterectomy without the removal of their ovaries. Hence, they were receiving estrogen replacement therapy without a progestational agent.

The testosterone gel was generally well tolerated. In Group I, one subject reported acne, one reported increased hair growth, and one experienced mild leg swelling. In Group II, one subject reported mild erythema at the gel application site, one reported oiliness of skin, two reported acne, four reported breast tenderness, and three reported vaginal spotting. There were no significant changes in hemoglobin, AST, and ALT during treatment.

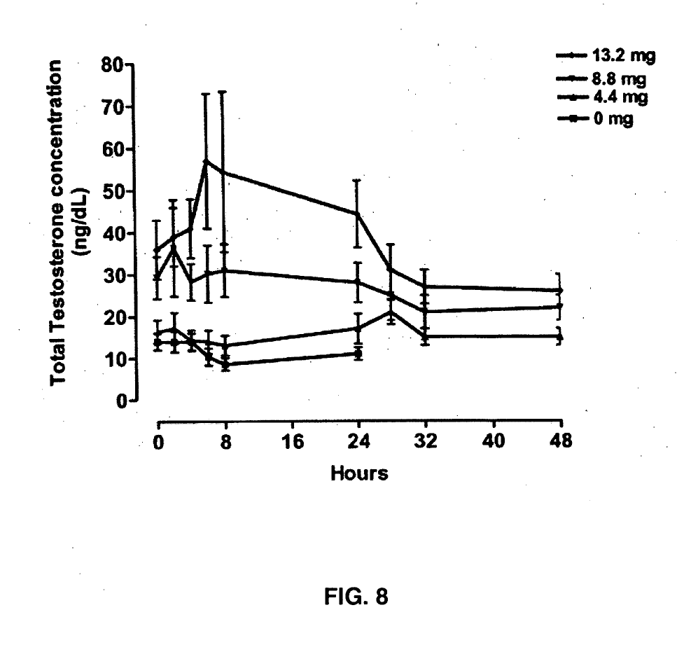

Serum total testosterone concentrations were low at baseline. In both groups of women, after application of the gel serum testosterone concentrations increased in proportion to the administered dose, as shown in FIGS. 8-11. Serum total testosterone concentrations on days 2, 15, and 29 at the beginning of each treatment period were not significant different from baseline levels regardless of the testosterone dose, demonstrating that the seven-day washout period was adequate in preventing any carryover effect, as shown in Table 22.

TABLE 22
Total Testosterone Concentrations (mean ± SEM) at the Beginning of
Each Treatment Period in Postmenopausal Women

| | | | |
|---|---|---|---|
| Baseline | Day 15 | Day 29 | P |

Postmenopausal Women Receiving Estrogen Replacement Therapy

| | | | | |
|---|---|---|---|---|
| Total Testosterone (ng/dL) | 9 ± 2 | 8.5 ± 2 | 10 ± 3 | 0.836 |
| Free Testosterone (pg/ml) | 5 ± 1 | 7 ± 2 | 7 ± 2 | 0.457 |

Postmenopausal Women Not Receiving Estrogen Replacement Therapy

| | | | | |
|---|---|---|---|---|
| Total Testosterone (ng/dL) | 12 ± 2 | 12 ± 2 | 13 ± 2 | 0.928 |
| Free Testosterone (pg/ml) | 6 ± 1 | 7 ± 1 | 5 ± 1 | 0.857 |

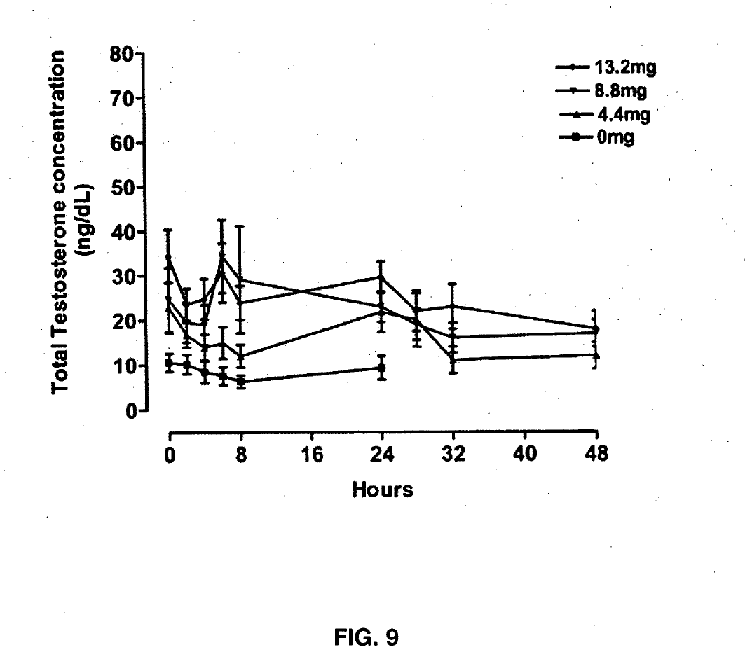

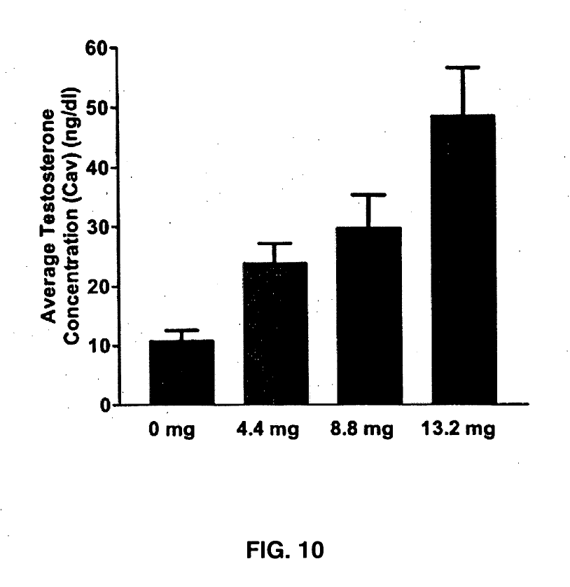

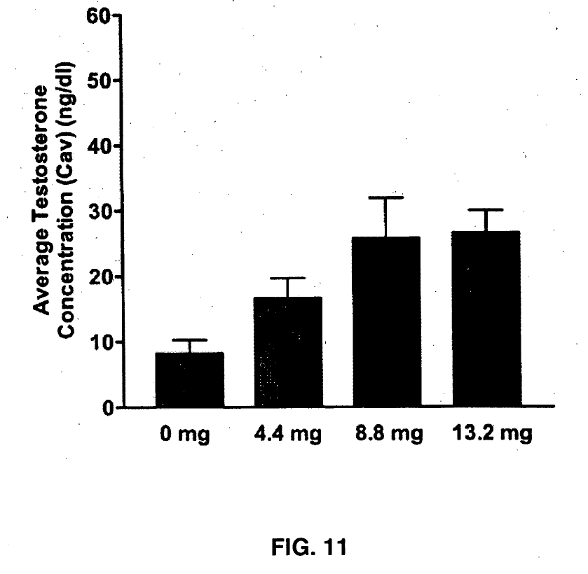

The analysis of variance revealed a significant testosterone dose effect on serum total testosterone concentrations (P=0.000001); however, there was no significant effect of estrogen replacement (P=0.11) on serum testosterone concentrations (FIGS. 8-11). In women who were receiving estrogen as well as in those who were not receiving estrogen, serum total testosterone concentrations at all doses were significantly different from one another at a joint significance level of 0.05 with the mean testosterone levels in numerical order by increasing dose level. Thus, the average serum testosterone concentrations were higher during treatment with 8.8 and 13.2 mg doses than at baseline or during administration of 4.4 mg testosterone gel (FIGS. 10 and 11). Administration of 8.8 and 13.2 mg doses increased serum total testosterone concentrations into the mid- to mid-high range, respectively, for healthy, menstruating women. Overall, serum total testosterone concentrations did not change significantly during the 24-hour sampling period at any of the testosterone doses (FIGS. 8 and 9). Serum total testosterone concentrations, however, started to decline between 24 and 36 hours after gel application (FIGS. 4 and 5).

There was no significant effect of estrogen therapy on serum testosterone concentrations at any time point during the 24-hour period (P=0.11). There was no significant interaction effect of testosterone dose and estrogen replacement status on serum total testosterone levels (P=0.82); this implies that there was no evidence of nonparallelism between the dose response curves of testosterone dose and serum testosterone concentrations in women who were on estrogen and those who were not.

The average (C_{average}), maximum (Cₘₐₓ), and minimum (Cₘᵢₙ) total testosterone concentrations during the 24-hour gel application period were significantly higher during administration of the 13.2 mg dose than at baseline or during administration of the 4.4 mg dose. There were no significant differences, however, in C_{average}, Cₘₐₓ, or Cₘᵢₙ between women who were on estrogen replacement and those who were not on estrogen replacement therapy, as shown in Table 23.

TABLE 23
Pharmacokinetic Parameters (mean ± SEM) Derived From Total
Testosterone Concentrations After Administration of 0, 4.4, 8.8, or
13.2 mg of Testosterone Gel in Postmenopausal Women

| | | | | | |
|---|---|---|---|---|---|
| Testosterone | | | | | |
| Dose | 0 mg | 4.4 mg | 8.8 mg | 13.2 mg | P |

Postmenopausal Women Not Receiving Estrogen Therapy

| | | | | | |
|---|---|---|---|---|---|
| Cₘₐₓ (ng/dL) | 18.2 ± 2.3 | 37.1 ± 5.3 | 46.6 ± | 71.0 ± | <0.000001 |
| | | | 9.4 | 11.5 | |
| Cₘᵢₙ (ng/dL) | 7.5 ± 2.1 | 15.2 ± 3.0 | 20.4 ± | 27.5 ± | <0.000001 |
| | | | 4.7 | 6.3 | |
| Cₐᵥ (ng/dL) | 10.8 ± 1.7 | 23.9 ± 3.3 | 29.8 ± | 48.6 ± | <0.000001 |
| | | | 5.5 | 8.0 | |
| Tₘₐₓ (h) | 4.3 ± 1.7 | 9.9 ± 2.1 | 9.4 ± | 7.5 ± | 0.2 |
| | | | 2.1 | 3.1 | |
| Tₘᵢₙ (h) | 7.9 ± 2.7 | 7.1 ± 3.2 | 7.4 ± | 5.2 ± | 0.32 |
| | | | 3.2 | 4.7 | |
| AUC₍₀₋₂₄₎ | 258.0 ± 35.4 | 574 ± 85.7 | 715 ± | 1170 ± | <0.000001 |
| (h.ng.dL) | | | 128 | 286 | |
| ΔCₐᵥ (ng/dL) | — | 13.1 ± 5.0 | 19.0 ± | 37.9 ± | — |
| | | | 11.6 | 9.3 | |
| Flux Index | 1.1 ± 0.6 | 0.8 ± 0.5 | 0.8 ± | 0.8 ± | — |
| | | | 0.4 | 0.4 | |

Postmenopausal Women Receiving Estrogen Therapy

| | | | | | |
|---|---|---|---|---|---|
| Cₘₐₓ (ng/dL) | 29.0 ± 6.1 | 27.8 ± 6.1 | 45.8 ± | 43.5 ± | <0.000001 |
| | | | 12.2 | 7.2 | |
| Cₘᵢₙ (ng/dL) | 5.7 ± 1.4 | 9.0 ± 1.8 | 11.1 ± | 16.6 ± | <0.000001 |
| | | | 1.5 | 2.4 | |
| Cₐᵥ (ng/dL) | 8.3 ± 2.0 | 16.6 ± 3.1 | 25.8 ± | 26.6 ± | <0.000001 |
| | | | 6.1 | 3.4 | |
| Tₘₐₓ (h) | 11.5 ± 3.6 | 10.7 ± 3.2 | 10.5 ± | 4.4 ± | 0.2 |
| | | | 2.6 | 2.2 | |
| Tₘᵢₙ (h) | 5.8 ± 0.7 | 5.8 ± 0.6 | 6.0 ± | 5.5 ± | 0.32 |
| | | | 2.0 | 0.8 | |
| AUC₍₀₋₂₄₎ | 198.0 ± 47.9 | 398.0 ± 73.2 | 618.0 ± | 639.0 ± | <0.000001 |
| (h.ng.dL) | | | 147.0 | 81.4 | |
| ΔCₐᵥ (ng/dL) | — | 8 ± 2 | 17.5 ± | 18.4 ± | — |
| | | | 5.8 | 2.1 | |
| Flux Index | 3.3 ± 0.5 | 1.1 ± 0.1 | 1.2 ± | 1.0 ± | — |
| | | | 0.2 | 0.2 | |

As set forth in Table 23, Tₘₐₓ and Tₘᵢₙ were not significantly different among the three doses that were tested. The average increment in serum total testosterone concentrations above baseline (delta-C_{average}) was proportional to the administered dose.

Serum free testosterone concentrations were low at baseline in both groups of women. Serum free testosterone concentrations were not significantly different from baseline levels at the beginning of each treatment period regardless of the testosterone dose (Table 22), confirming that the seven-day washout period between doses was adequate in preventing any carryover effect.

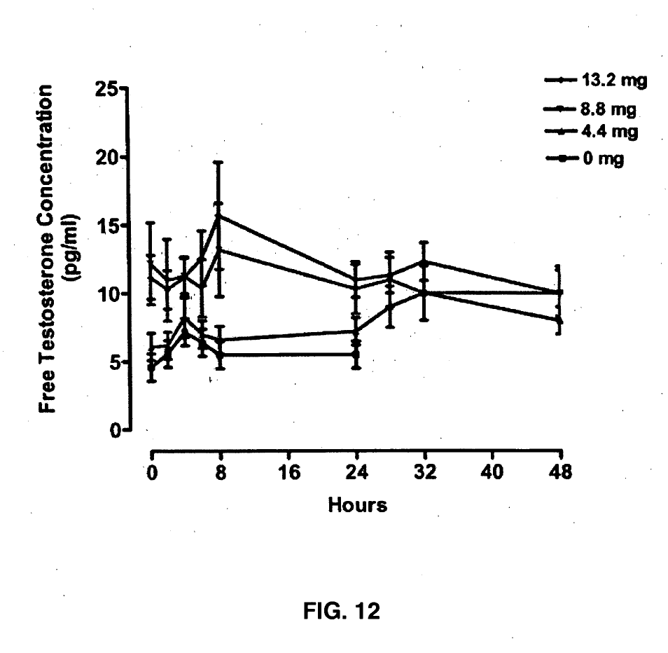

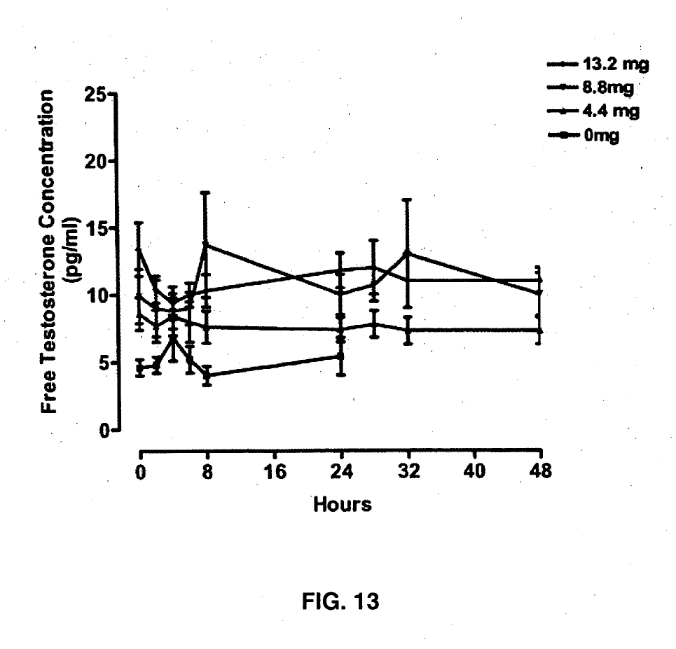

After application of the testosterone gel, serum free testosterone concentrations increased in proportion to the administered dose, as shown in FIGS. 12 and 13. A very significant testosterone dose effect (P=0.0003) on free testosterone concentrations was observed. There was no significant effect, however, of estrogen replacement (P=0.96) on serum free testosterone concentration. The Tukey-Kramer multiple comparison analysis revealed that serum free testosterone concentrations after administration of the 8.8 and 13.2 mg doses were significantly higher than at baseline or during administration of the 4.4 mg dose, but not significantly different from each other at the joint 5% significance level. There was no estrogen treatment effect on serum free testosterone concentrations at any time point during the 24-hour period at any dose of testosterone gel. Also, serum free testosterone concentrations were not significantly different at different time points during the 24-hour sampling period at any of the doses tested.

The C_{average}, Cₘₐₓ, and Cₘᵢₙ for free testosterone concentrations during the 24-hour gel application period increased in proportion to the administered dose and were significantly higher in those receiving the 13.2 mg dose than at baseline or during administration of the 4.4, mg dose, as shown in Table 24.

TABLE 24
Pharmacokinetic Parameters (mean ± SEM) Derived From Free
Testosterone Concentrations After Administration of 0, 4.4, 8.8, or
13.2 mg of Testosterone Gel in Postmenopausal Women

| | | | | | |
|---|---|---|---|---|---|
| Testosterone | | | | | |
| Dose | 0 mg | 4.4 mg | 8.8 mg | 13.2 mg | P |

Postmenopausal Women Not Receiving Estrogen Therapy

| | | | | | |
|---|---|---|---|---|---|
| Cₘₐₓ (pg/ml) | 9.4 ± 1.5 | 11.6 ± 1.7 | 15.6 ± | 19.2 ± | <0.00002 |
| | | | 3.3 | 4.0 | |
| Cₘᵢₙ (pg/ml) | 4.1 ± 0.7 | 5.9 ± 1.0 | 7.6 ± | 8.1 ± | <0.000001 |
| | | | 1.2 | 1.1 | |
| Cₐᵥ (pg/ml) | 5.9 ± 0.9 | 8.4 ± 1.2 | 11.5 ± | 12.8 ± | <0.000001 |
| | | | 2.5 | 2.1 | |
| Tₘₐₓ (h) | 5.2 ± 1.6 | 8.9 ± 2.5 | 7.5 ± | 4.2 ± | 0.14 |
| | | | 2.2 | 0.9 | |
| Tₘᵢₙ (h) | 2.8 ± 1.8 | 4.3 ± 1.8 | 7.7 ± | 7.5 ± | 0.29 |
| | | | 2.6 | 2.7 | |
| AUC₍₀₋₂₄₎ | 140.0 ± 5.8 | 201.0 ± 98.4 | 276.0 ± | 307.0 ± | <0.000001 |
| (h.pg.ml) | | | 58.8 | 49.9 | |
| ΔCₐᵥ (pg/ml) | — | 2.5 ± 1.4 | 5.6 ± | 6.9 ± | — |
| | | | 6.0 | 2.2 | |
| Flux Index | 0.99 ± 0.7 | 0.7 ± 0.5 | 0.6 ± | 0.7 ± | — |
| | | | 0.3 | 0.3 | |

Postmenopausal Women Receiving Estrogen Therapy

| | | | | | |
|---|---|---|---|---|---|
| Cₘₐₓ (pg/ml) | 9.3 ± 1.8 | 11.3 ± 1.6 | 16.2 ± | 15.4 ± | <0.00002 |
| | | | 3.8 | 1.7 | |
| Cₘᵢₙ (pg/ml) | 2.9 ± 0.6 | 5.2 ± 0.6 | 5.8 ± | 7.2 ± | <0.000001 |
| | | | 0.8 | 0.7 | |
| Cₐᵥ (pg/ml) | 5.0 ± 0.7 | 7.6 ± 1.0 | 11.1 ± | 10.8 ± | <0.000001 |
| | | | 1.9 | 1.0 | |
| Tₘₐₓ (h) | 7.8 ± 2.5 | 7.8 ± 2.6 | 8.9 ± | 9.8 ± | 0.14 |
| | | | 2.4 | 3.5 | |
| Tₘᵢₙ (h) | 7.5 ± 2.6 | 8.9 ± 2.3 | 4.2 ± | 5.1 ± | 0.29 |
| | | | 2.1 | 0.7 | |
| ΔAUC₍₀₋₂₄₎ | 119.0 ± 5.5 | 182.0 ± 22.9 | 266.0 ± | 258.0 ± | <0.000001 |
| (h.pg.ml) | | | 46.3 | 23.7 | |
| ΔCₐᵥ (pg/ml) | — | 2.7 ± 0.7 | 6.2 ± | 5.8 ± | − |
| | | | 1.9 | 0.6 | |

The average increment in serum free testosterone above baseline was related to testosterone dose. Thus, delta C_{average} was greater during administration of the 13.2 mg dose than during baseline sampling or during administration of 4.4 mg dose. C_{average}, Cₘₐₓ, and Cₘᵢₙ were not significantly different at any dose between women who were on estrogen replacement and those who were not on estrogen replacement therapy.

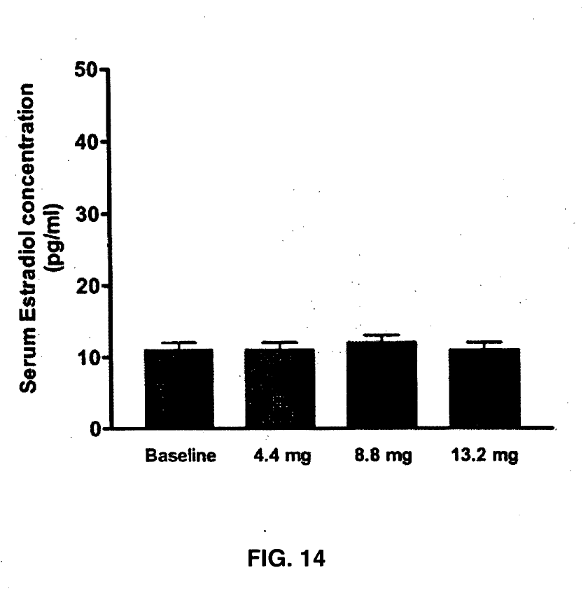

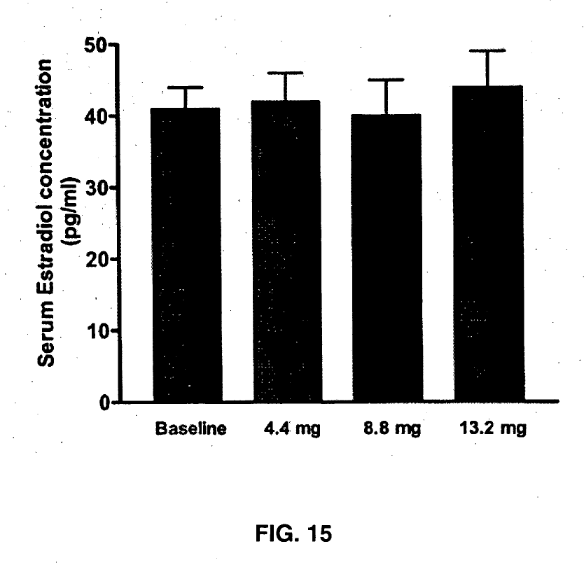

As expected, serum estradiol levels were higher at baseline (41±3 vs. 11±1 pg/mL) and at all time points during testosterone treatment in women who were on estrogen replacement therapy than in those who were not (P<0.000001) (FIGS. 14 and 15). Serum estradiol concentrations did not change significantly in any of the groups after testosterone gel application (P=0.37). There was no significant interaction effect of testosterone and estrogen treatment on serum estradiol concentrations (P=0.59).

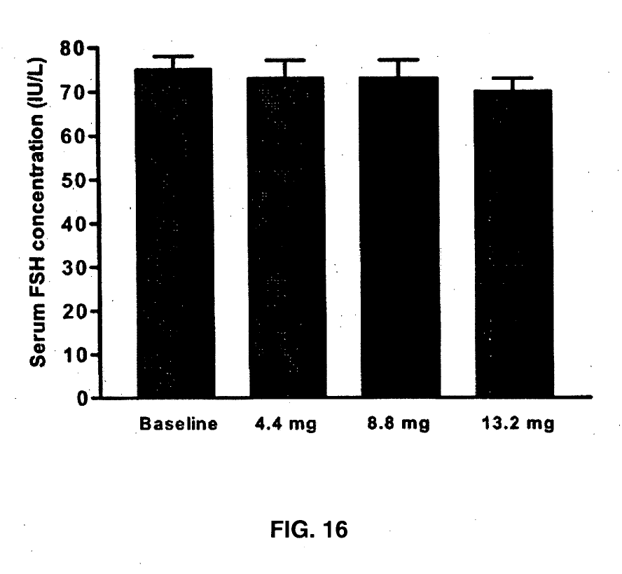

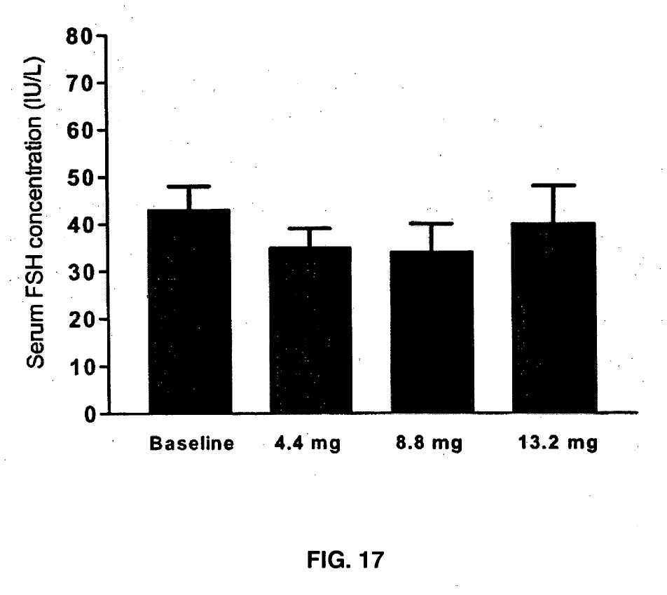

At baseline, serum FSH concentrations were significantly lower in women who were receiving estrogen replacement therapy than in those who were not (43±5 vs. 74±7 U/L) (FIGS. 16 and 17). There was a significant difference in FSH levels in subjects receiving estrogen compared to those who did not (P=0.000001). There was no significant effect, however, of testosterone dose in either treatment group (P=0.33).

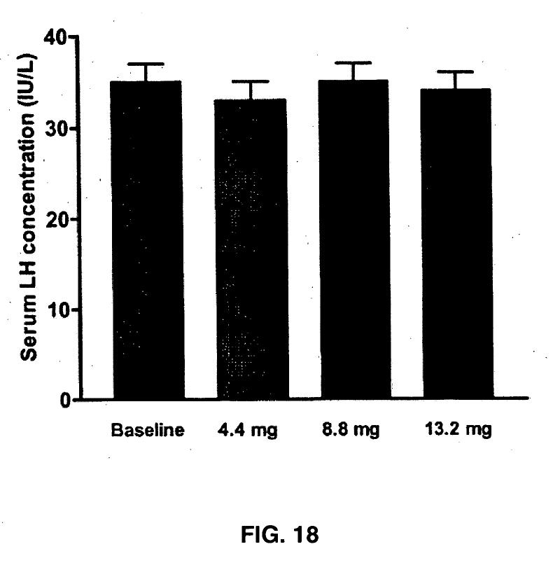

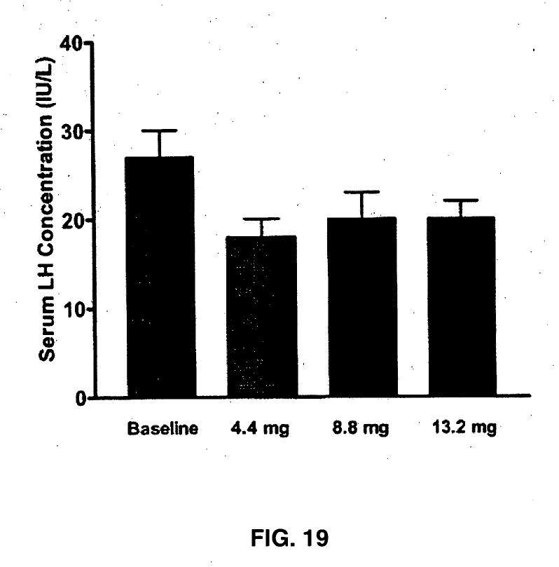

Serum LH concentrations were significantly lower in women who were receiving estrogen replacement therapy than in those who were not (27±3 vs. 35±2 U/L; P=0.001) (FIGS. 18 and 19). During testosterone administration, serum LH concentrations were significantly different between those individuals receiving estrogen and those who did not (P=0.000001). There were no significant differences, however, in LH levels during administration of different testosterone doses in either group (P=0.97).

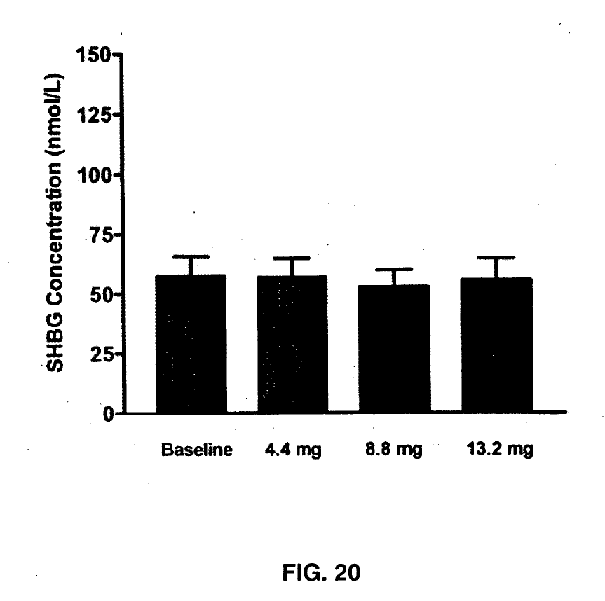

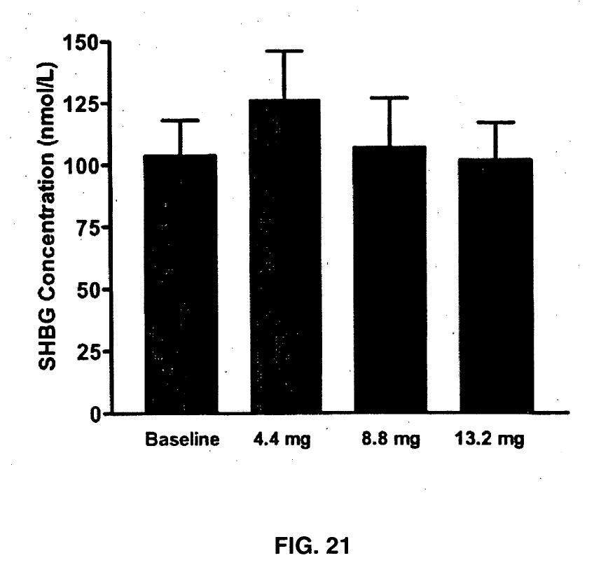

Serum sex hormone binding globulin (SHBG) concentrations were numerically higher in women who were receiving estrogen replacement therapy; however, the differences in SHBG concentrations between these two groups did not achieve statistical significance (P=0.21) (P=0.63) (FIGS. 20 and 21). Serum SHBG levels did not change significantly during administration of testosterone at any dose in either group (P=0.76).

This example demonstrates that treatment with testosterone gel in postmenopausal women with low testosterone levels was associated with a dose-dependent increase in serum total and free testosterone concentrations. Transdermal application of the 8.8 and 13.2 mg testosterone gel daily raised and maintained serum total and free testosterone levels into the mid- to high-mid range for healthy menstruating women. The flatness of the total and free testosterone concentration profiles is consistent with relatively uniform testosterone delivery during the 24-hour sampling period after testosterone gel application. These data justify once daily application of the gel. Hormone replacement therapy with conjugated equine estrogen and medroxyprogesterone acetate did not significantly affect the total or free testosterone concentrations over the treatment period.

### Example 7

### Dosage of Testosterone in a Female after Bilateral Oophorectomy

In one embodiment of the present invention, the methods, kits, combinations, and compositions are comprised of a percutaneously deliverable testosterone formulation. In this example, testosterone is formulated as a gel for transdermal administration as described above in Table 4.

In a prophetic example, 24 premenopausal women who have undergone bilateral oophorectomy are randomized to receive: (a) 0.17 g/day of RELIBRA, which delivers 1.7 mg/day of testosterone to the skin of which about 0.1 mg, is absorbed, for 30 days; or (b) 0.25 g/day of RELIBRA, which delivers 2.5 mg/day of testosterone to the skin of which about 0.15 mg is absorbed, for 30 days; or (c) 0.5 g/day of RELIBRA, which delivers 5 mg/day of testosterone to the skin of which about 0.3 mg is absorbed, for 30 days; or (d) a gel containing a placebo for 30 days. The gel is rubbed onto the clean dry skin of the upper outer thigh and hip once daily. Following application, the gel is allowed to air dry. The patient washes her hands.

Applicants expect that from a physiological perspective, all test parameters will show an improvement in female sexual dysfunction over the placebo. Accordingly, Applicant expects that RELIBRA can be applied to improve female sexual dysfunction as compared to placebo in premenopausal women who have undergone a bilateral oophorectomy.

### Example 8

### Dosage of Testosterone and Methyltestosterone in a Female after Bilateral Oophorectomy

In one embodiment of the present invention, the methods, kits, combinations, and compositions are comprised of a percutaneously deliverable testosterone formulation, and an orally deliverable methyltestosterone formulation. In this example, testosterone is formulated as a gel for transdermal administration as described above in Table 4, and methyltestosterone is formulated as a capsule for oral administration and each dosage unit contains 10 mg of methyltestosterone.

In a prophetic example, 24 premenopausal women who have undergone bilateral oophorectomy are randomized to receive a daily oral dose of 10 mg or 50 mg methyltestosterone for 30 days, plus: (a) 0.17 g/day of RELIBRA, which delivers 1.7 mg/day of testosterone to the skin of which about 0.1 mg, is absorbed, for 30 days; or (b) 0.25 g/day of RELIBRA, which delivers 2.5 mg/day of testosterone to the skin of which about 0.15 mg is absorbed, for 30 days; or (c) 0.5 g/day of RELIBRA, which delivers 5 mg/day of testosterone to the skin of which about 0.3 mg is absorbed, for 30 days; or (d) a gel containing a placebo for 30 days. The gel is rubbed onto the clean dry skin of the upper outer thigh and hip once daily. Following application, the gel is allowed to air dry. The patient washes her hands.

Applicants expect that from a physiological perspective, all test parameters will show an improvement in female sexual dysfunction over the placebo. Accordingly, Applicant expects that RELIBRA can be administered in conjunction with methyltestosterone to improve female sexual dysfunction as compared to placebo in premenopausal women who have undergone a bilateral oophorectomy.

### Example 9

### Dosage of Testosterone and Estrogen in a Female after Bilateral Oophorectomy

In one embodiment of the present invention, the methods, kits, combinations, and compositions are comprised of a percutaneously deliverable testosterone formulation, and a non-orally deliverable estrogen. In this example, testosterone is formulated as a gel for transdermal administration as described above in Table 4, and estradiol is formulated as a gel for transdermal administration as described above in Table 6.

In a prophetic example, 24 premenopausal women who have undergone bilateral oophorectomy are randomized to receive a daily dose of 5 g or 10 g ESTROGEL for 30 days, plus: (a) 0.17 g/day of RELIBRA, which delivers 1.7 mg/day of testosterone to the skin of which about 0.1 mg, is absorbed, for 30 days; or (b) 0.25 g/day of RELIBRA, which delivers 2.5 mg/day of testosterone to the skin of which about 0.15 mg is absorbed, for 30 days; or (c) 0.5 g/day of RELIBRA, which delivers 5 mg/day of testosterone to the skin of which about 0.3 mg is absorbed, for 30 days; or (d) a gel containing a placebo for 30 days. The gel is rubbed onto the clean dry skin of the upper outer thigh and hip once daily. Following application, the gel is allowed to air dry. The patient washes her hands.

Applicants expect that from a physiological perspective, all test parameters will show an improvement in female sexual dysfunction over the placebo. Accordingly, Applicant expects that RELIBRA can be administered in conjunction with estradiol to improve female sexual dysfunction as compared to placebo in premenopausal women who have undergone a bilateral oophorectomy.

TABLE 25
Combination Testosterone and Estrogen Gel

| Substance | Amount (w/w) per 100 g of Gel | |
|---|---|---|
| Testosterone | 1 | g (or about 0.5 g) |
| 17-beta-oestradiol | 0.06 | g (or about 0.10 g) |
| Carbopol 980 | 1 | g |
| Triethanolamine | 1.35 | g |
| Isopropyl myristate | 0.50 | g |
| 0.1 N NaOH | 4.72 | g |
| Ethanol (95% v/v) | 72.5 | g |
| Purified Water (qsf) | 100 | g |

The gel is rubbed onto the clean dry skin of the upper outer thigh and hip once daily. Following application, the gel is allowed to air dry. The patient washes her hands. Application of the gel results in an increased testosterone level having a desirable pharmacokinetic profile similar to that in normal women. The gel is thus useful for treating a number of conditions or diseases in women.

### Example 10

### In Vitro Skin Penetration Test Method

Skin penetration of testosterone and the other therapeutic agents of the present invention can be determined using the following method. A diffusion cell is used with either hairless mouse skin or human cadaver skin.

The composition is applied to the skin and rubbed onto a predetermined area to cause uniform contact with the skin. The resulting composition/skin is placed composition side up across the orifice of the lower portion of the diffusion cell. The diffusion cell is assembled and the lower portion is filled with 10 mL of warm (32° C.) receptor fluid so that the receptor fluid is in contact with the skin. The receptor fluid is stirred using a magnetic stirrer. The sampling port is covered except when in use.

The cell is then placed in a constant temperature (32° C.) and humidity (50% relative humidity) chamber. The receptor fluid is stirred by means of a magnetic stirrer throughout the experiment to assure a uniform sample and a reduced diffusion barrier on the dermal side of the skin. The entire volume of receptor fluid is withdrawn at specified time intervals and immediately replaced with fresh fluid. The withdrawn fluid is filtered through a 0.45 μM filter then analyzed for the testosterone or the therapeutic agent using high performance liquid chromatography. The cumulative amount of testosterone or therapeutic agent penetrating the skin and the flux rate is calculated.

The contents of all cited references throughout this application are hereby expressly incorporated by reference. The practice of the present invention will employ, unless otherwise indicated, conventional techniques of pharmacology and pharmaceutics, which are within the skill of the art.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed is:

1. A method of treating, preventing or reducing the risk of developing a testosterone-deficient disorder in a female subject in need thereof, comprising:
(i) identifying a female subject having, or at risk of developing, a testosterone-deficient disorder;
(ii) administering an amount of a hydroalcoholic gel pharmaceutical composition to an area of skin of the subject, which delivers a therapeutically-effective amount of testosterone to the blood serum of the subject such that the testosterone-deficient disorder is improved or the risk of developing a testosterone-deficient disorder is reduced, wherein the composition comprises:
a. about 0.1% to about 10% (w/w) testosterone, or a salt, ester, amide, enantiomer, isomer, tautomer, prodrug, or derivative thereof;
b. about 30% to about 98% (w/w) alcohol selected from the group consisting of ethanol or isopropanol;
c. about 0.1% to about 5% (w/w) penetration enhancer;
d. about 0.1% to about 5% (w/w) of a gelling agent; and
e. the balance purified water;
wherein the composition is capable of releasing the testosterone to the skin at a rate and duration that raises testosterone blood serum concentration to at least about 3 pg testosterone/ml blood serum within about 24 hours after administration.

2. The method of claim 1, wherein the composition comprises about 1% testosterone.

3. The method of claim 1, wherein the composition comprises about 67% alcohol.

4. The method of claim 1, wherein the penetration enhancer is isopropyl myristate.

5. The method of claim 1, wherein the composition comprises about 0.5% isopropyl myristate.

6. The method of claim 1, wherein the gelling agent is selected from the group consisting of polyacrylic acid and carboxymethylcellulose.

7. The method of claim 1, wherein the gelling agent is polyacrylic acid present in an amount of about 1% weight to weight of the composition.

8. The method of claim 1, wherein the composition comprises:
a. about 1.0% testosterone;
b. about 0.9% CARBOPOL®;
c. about 0.5% isopropyl myristate;
d. about 67% ethanol; and
e. the balance purified water;
wherein the percentages are on a weight to weight basis of the composition.

9. The method of claim 1, wherein for each about 0.1 gram per day application of the composition to the skin, an increase of at least about 5 ng/dl in serum testosterone concentration results in the subject.

10. The method of claim 1, wherein the composition is provided to the subject for daily administration in about a 0.1 g to about a 10 g dose.

11. The method of claim 1, wherein the amount of the composition is a 0.44 g dose delivering about 0.44 mg to about 44 mg of testosterone to the skin.

12. The method of claim 1, wherein the amount of the composition is a 1.32 g dose delivering about 1.32 mg to about 132 mg of testosterone to the skin.

13. The method of claim 1, wherein the amount of the composition is a 1.32 g dose delivering 6.6 mg to about 13.2 mg of testosterone to the skin.

14. The method of claim 11, wherein the amount of the composition is either a 0.44 g dose delivering about 4.4 mg of testosterone to the skin or a 0.88 g dose delivering about 8.8 mg of testosterone to the skin.

15. The method of claim 14, wherein the subject is premenopausal with a below-normal free (unbound) testosterone concentration less than 0.5 ng/dl.

16. The method of claim 14, wherein the subject is postmenopausal with a below-normal free (unbound) testosterone concentration less than 0.5 ng/dl.

17. The method of claim 1, wherein the composition is provided to the subject in one or more packets.

18. The method of claim 17, wherein the packet comprises a polyethylene liner between the composition and inner surface of the packet.

19. The method of claim 1, wherein the composition is administered once, twice, or three times a day.

20. The method of claim 1, wherein the composition further comprises about 0.01% to about 69% of a therapeutic agent comprising an agent that inhibits the synthesis of the sex hormone binding globulin, a progesterone, a progestin, or an estrogenic hormone.

21. The method of claim 20, wherein said therapeutic agent comprises about 1% to about 10% of the composition.

22. The method of claim 20, wherein said therapeutic agent is progesterone.

23. The method of claim 22, wherein serum blood level of progesterone is raised to at least about 1 ng progesterone/ml blood serum within about 24 hours after administration.

24. The method of claim 20, wherein said therapeutic agent is estrogen.

25. The method of claim 24, wherein the serum-blood level of estrogen is raised to at least 60 pg estrogen/ml blood serum within about 24 hours after administration.

26. The method of claim 20, wherein said therapeutic agent is methyltestosterone.

27. The method of claim 20, wherein said therapeutic agent is estradiol.

28. A method for improving sexual performance in a female subject in need thereof, comprising administering to the subject:
(a) an amount of a hydroalcoholic gel pharmaceutical composition to an area of skin of the subject, which delivers a therapeutically-effective amount of testosterone to the blood serum of the subject, wherein the composition comprises:
i. about 0.1% to about 10% (w/w) testosterone, or a salt, ester, amide, enantiomer, isomer, tautomer, prodrug, or derivative thereof;
ii. about 30% to about 98% (w/w) alcohol selected from the group consisting of ethanol or isopropanol;
iii. about 0.1% to about 5% (w/w) penetration enhancer;
iv. about 0.1% to about 5% (w/w) of a gelling agent; and
v. the balance water; and
(b) an amount of an agent for improving sexual performance.

29. The method of claim 28, wherein the agent is selected from the group consisting of: an agent that causes vasodilatation, a nitrate, an alpha-blocker, a naturally occurring prostaglandin, an endothelial-derived relaxation factor, a phosphodiesterase inhibitor, or combinations thereof.
